(19) United States
(12) Patent Application Publication
(10) Pub. No.: US 2011/0009453 A1
(43) Pub. Date: Jan. 13, 2011

(54) S1P3 RECEPTOR INHIBITORS FOR TREATING INFLAMMATION

(76) Inventors: John E. Donello, Dana Point, CA (US); Mohammed I. Dibas, Mission Viejo, CA (US)

Correspondence Address: ALLERGAN, INC., 2525 DUPONT DRIVE, T2-7H, IRVINE, CA, 92612-1599, US

(21) Appl. No.: 12/922,629

(22) PCT Filed: Mar. 16, 2009
(86) PCT No.: PCT/US09/37219

§ 371 Date: Sep. 14, 2010

Related U.S. Application Data
(60) Provisional application No. 61/037,250, filed on Mar. 17, 2008.

(51) Int. Cl. A61K 31/444; A61K 31/4439; A61K 31/403

(52) U.S. Cl. 514/333; 514/339; 514/419

(57) ABSTRACT

Disclosed herein are compositions and methods for treating inflammation using S1 P3 receptor inhibitors.

### CROSS REFERENCE

This application claims priority to U.S. Provisional Application serial number to 61/037,250, filed Mar. 17, 2008, which is hereby incorporated by reference in its entirety.

### DETAILED DESCRIPTION OF THE INVENTION

Disclosed herein is a method for treating inflammation of human tissues and organs, the method comprising administering to a patient in need of such treatment an S1P3 receptor inhibitor.

### S1P3 Receptor

Sphingosine-1-phosphate (“S1P”) is an important chemical messenger that can activate particular cell surface transmembrane G-protein coupled receptors known as endothelial gene differentiation (“Edg”) receptors.

There are five known SIP receptors activated by SIP: S1P1, also known as Edg 1 (human Edg-1, GenBank Accession No. AF233365); S1P2, also known as Edg 5 (human Edg-5, GenBank Accession No. AF034780); S1P3, also known as Edg 3 (human Edg-3, GenBank Accession No. X83864); S1P4, also known as Edg 6 (human Edg-6, GenBank Accession No. AF000479); and S1P5, also known as Edg 8 (human Edg-8, GenBank Accession No. AF317676).

The method of the present invention treats inflammation by administering compounds that inhibit the S1P3 receptor. In one embodiment, the method administers compounds that selectively inhibit the S1P3 subtype as compared to at least one other S1P subtype.

### S1P3 Receptor Inhibitors

A compound is an “S1P3 receptor inhibitor” if it inhibits, partially or completely, the cellular response caused by binding of S1P or other ligand to the S1P3 receptor.

S1P3 is a G-protein coupled receptor (GPCR). When a ligand binds to that receptor it induces a conformational shift, causing GDP to be replaced by GTP on the α-subunit of the associated G-proteins and subsequent release of the G-proteins into the cytoplasm. The α-subunit then dissociates from the βγ-subunit and each subunit can then associate with effector proteins, activating second messengers, and leading to a cellular response. The process is referred to as S1P cell signaling.

One example of a cellular response is the accumulation of cAMP. The effect of an inhibitor on this response can be measured by well-known techniques in the art. One example is radioimmunoassay and the [γ-³⁵S]GTP binding assay, illustrated in U.S. Patent Application Publication No. 2005/0222422 and No. is 2007/0088002 to assay S1P agonists (the disclosures of both these publications are incorporated by reference). To evaluate a compound for its potential as an inhibitor, one can measure cAMP accumulation by radioimmunoassay after incubating S1P (or S1P receptor agonist) in the presence of a test compound and cells expressing the S1P3 receptor; if the compound is an inhibitor, it will reduce the activation of S1P3 by S1P, which can be measured as reduced cAMP accumulation.

Another method of determining if a compound is an S1P3 receptor inhibitor is with a FLIPR assay. An example of this method is described in U.S. patent application Ser. No. 11/675,168, the contents of which are incorporated herein by reference. According to that application, compounds may be assessed for their ability to activate or block activation of the human S1P3 receptor in T24 cells stably expressing the human S1P3 receptor. In this assay ten thousand cells/well are plated into 384-well poly-D-lysine coated plates one day prior to use. The growth media for the S1P3 receptor expressing cell line is McCoy's 5A medium supplemented with 10% charcoal-treated fetal bovine serum (FBS), 1% antibiotic-antimycotic and 400 μg/ml geneticin. On the day of the experiment, the cells are washed twice with Hank's Balanced Salt Solution supplemented with 20 mM HEPES (HBSS/Hepes buffer). The cells are then dye loaded with 2 uM Fluo-4 diluted in the HBSS/Hepes buffer with 1.25 mM Probenecid and incubated at 37° C. for 40 minutes. Extracellular dye is removed by washing the cell plates four times prior to placing the plates in the FLIPR (Fluorometric Imaging Plate Reader, Molecular Devices). Ligands are diluted in HBSS/Hepes buffer and prepared in 384-well microplates. The positive control, S1P, is diluted in HBSS/Hepes buffer with 4 mg/ml fatty acid free bovine serum albumin. The FLIPR transfers 12.5 μl from the ligand microplate to the cell plate and takes fluorescent measurements for 75 seconds, taking readings every second, and then for 2.5 minutes, taking readings every 10 seconds. Drugs are tested over the concentration range of to 0.61 nM to 10,000 nM. Data for Ca⁺² responses is obtained in arbitrary fluorescence units and not translated into Ca⁺² concentrations. IC₅₀ values are determined through a linear regression analysis using the Levenburg Marquardt algorithm.

S1P3 receptor inhibitors include S1P3 receptor antagonists and S1P3 is receptor inverse agonists, as long as they inhibit, partially or completely, S1P cell signaling.

S1P3 receptor inhibitors may be selective for the S1P3 receptor or they may inhibit SIP cell signaling at more than one of the SIP receptor subtypes. An inhibitor is selective for the S1P3 receptor compared to another S1P subtype if the inhibitor is more than 100 times as potent at inhibiting the S1P3 receptor than it is at inhibiting or activating the other S1P receptor subtype. For example, the IC₅₀ of hypothetical compound A in a FLIPR assay is 100 nM at the S1P3 receptor, >5000 nM at the S1P1 receptor, and 200 nM at the S1P5 receptor; compound A is selective for the S1P3 receptor compared to the S1P1 receptor but not compared to the S1P5 receptor. If, to take another example, the IC₅₀ of hypothetical compound B is 100 nM at the S1P3 receptor and EC₅₀ is 200 nM at the S1P1 receptor and >5000 at the S1P2 receptor, then compound B is selective for the S1P3 receptor compared to the S1P2 receptor but not the S1P1 receptor.

In one embodiment, the S1P3 receptor inhibitors are selective for the S1P3 receptor as compared to one receptor selected from the group consisting of the S1P1, S1P2, S1P4, and S1P5 receptors. In another embodiment, the S13P receptor inhibitors are selective for the S1P3 receptor as compared to two receptors selected from the group consisting of the S1P1, S1P2, S1P4, and S1P5 receptors. In another embodiment, the S13P receptor inhibitors are selective for the S1P3 receptor as compared to three receptors selected from the group consisting of the S1P1, S1P2, S1P4, and S1P5 receptors. In another embodiment, the S13P receptor inhibitors are selective for the S1P3 receptor as compared to S1P1, S1P2, S1P4, and S1P5 receptors.

S13P Receptor Inhibitors useful in the Method of the Invention

S1P3 receptor inhibitors useful in the method of the invention include those disclosed in U.S. patent application Ser. No. 11/675,168, No. 11/690,637, No. 12/013,239 (claiming priority from No. 60/884,470), and No. 11/850,756 (claiming priority from No. 60/824,807), and in U.S. Patent Application Publication No. 2005/0222422, No. 2007/0032459 and No. 2008/0025973. The disclosures of all the foregoing references are incorporated by reference.

### DEFINITIONS

In describing S13P receptor inhibitors useful in the invention, the following terms have the following meanings, unless otherwise indicated.

“Me” refers to methyl.

“Et” refers to ethyl.

“tBu” refers to t-butyl.

“iPr” refers to i-propyl.

“Ph” refers to phenyl.

“Alkyl” refers to a straight-chain, branched or cyclic saturated aliphatic hydrocarbon. The alkyl group may have 1 to 12 carbons; in other embodiments, it is a lower alkyl of from 1 to 7 carbons, or a lower alkyl from 1 to 4 carbons. Typical alkyl groups include methyl, ethyl, propyl, isopropyl, butyl, isobutyl, tertiary butyl, pentyl, hexyl and the like. The alkyl group may be optionally substituted with one or more substituents are selected from the group consisting of hydroxyl, cyano, alkoxy, ═O, ═S, NO₂, halogen, dimethyl amino and SH.

“Alkenyl” refers to a straight-chain, branched or cyclic unsaturated hydrocarbon group containing at least one carbon-carbon double bond. The alkenyl group may have 2 to 12 carbons; in other embodiments, it is a lower alkenyl of from 2 to 7 carbons, or a lower alkenyl of from 2 to 4 carbons. The alkenyl group may be optionally substituted with one or more substituents selected from the group consisting of hydroxyl, cyano, alkoxy, O, S, NO₂, halogen, dimethyl amino and SH.

“Alkynyl” refers to a straight-chain, branched or cyclic unsaturated hydrocarbon containing at least one carbon-carbon triple bond. The alkynyl group may have 2 to 12 carbons; in other embodiments, it is a lower alkynyl of from 2 to 7 carbons, or a lower alkynyl of from 2 to 4 carbons. The alkynyl group may be optionally substituted with one or more substituents selected from the group consisting of hydroxyl, cyano, alkoxy, O, S, NO₂, halogen, dimethyl amino and SH.

“Alkoxy” refers to an “O-alkyl” group.

“Aryl” refers to an aromatic group which has at least one ring having a conjugated pi electron system and includes carbocyclic aryl, heterocyclic aryl and biaryl groups. The aryl group may be optionally substituted with one or more substituents selected from the group consisting of halogen, trihalomethyl, is hydroxyl, SH, OH, NO₂, amine, thioether, cyano, alkoxy, alkyl, and amino.

“Alkaryl” (Alkylaryl) refers to an alkyl that is covalently joined to an aryl group. In one embodiment, the alkyl is a lower alkyl.

“Aryloxy” refers to an “O-aryl” group.

“Arylalkyloxy” refers to an “O-alkaryl” (O-alkylaryl) group.

“Carbocyclic aryl” refers to an aryl group wherein the ring atoms are carbon.

“Heterocyclic aryl” refers to an aryl group having from 1 to 3 heteroatoms as ring atoms, the remainder of the ring atoms being carbon. Heteroatoms include oxygen, sulfur, and nitrogen.

“Hydrocarbyl” refers to a hydrocarbon radical having only carbon and hydrogen atoms. The hydrocarbyl radical may have from 1 to 20 carbon atoms, or from 1 to 12 carbon atoms, or from 1 to 7 carbon atoms.

“Substituted hydrocarbyl” refers to a hydrocarbyl radical wherein one or more, but not all, of the hydrogen and/or the carbon atoms are replaced by a halogen, nitrogen, oxygen, sulfur or phosphorus atom or a radical including a halogen, nitrogen, oxygen, sulfur or phosphorus atom, e.g. fluoro, chloro, cyano, nitro, hydroxyl, phosphate, thiol, etc.

“Amide” refers to —C(O)—NH—R′, wherein R′ is alkyl, aryl, alkylaryl or hydrogen.

“Ester” refers to —C(O)—O—R′, wherein R′ is alkyl, aryl or alkylaryl.

“Carboxy” refers to —C(O)—O—H

“Thioamide” refers to —C(S)—NH—R′, wherein R′ is alkyl, aryl, alkylaryl or hydrogen.

“Thiol ester” refers to —C(O)—S—R′, wherein R′ is alkyl, aryl, alkylaryl or hydrogen.

“Amine” refers to a —N(R″)R′″ group, wherein R″ and R′″ are independently selected from the group consisting of alkyl, aryl, and alkylaryl.

“Thioether” refers to —S—R″, wherein R″ is alkyl, aryl, or alkylaryl.

“Sulfonyl” refers to —S(O)₂—R″″, wherein R″″ is alkyl, aryl, C(CN)═C-aryl, CH₂ CN, or alkyaryl.

“Sulfoxyl” refers to —S(O) —R″″, wherein R″″ is alkyl, alkenyl, alkynyl, aryl, or alkylaryl.

“Sulfonamidyl” refers to —S(O)—NR′(R″), wherein R′ and R″ are independently alkyl, alkenyl, alkynyl, aryl, or alkylaryl.

“Carbocyclic” refers to any ring, aromatic or non-aromatic, containing 1 to 12 carbon atoms.

“Heterocyclic” refers to any ring, aromatic or non-aromatic, containing 1 to 12 carbon atoms and 1 to 4 heteroatoms chosen from a group consisting of oxygen, nitrogen and sulfur.

### Indole-3-Carboxylic Acid Amide, Ester, Thioamide and Thiol Ester Compounds Bearing Aryl or Heteroaryl Groups

U.S. patent application Ser. No. 11/675,168 discloses S1P3 receptor antagonists having the following formula:

wherein

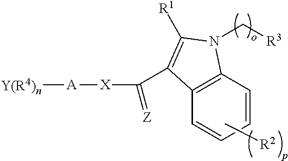

X is NR⁵, O, S;

Z is O or S;

n is 0 or an integer of from 1 to 4;

o is 0 or an integer of from 1 to 3;

p is 0 or an integer of from 1 to 4;

A is (C(R⁵)₂)ₘ, wherein
m is 0 or an integer of from 1 to 6;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl groups;

Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

R¹, R², R³, R⁴ are selected from the group consisting of hydrogen; straight or branched chain alkyl having 1 to 12 carbons; cycloalkyl having 3 to 6 carbons; alkenyl having 2 to 6 carbons and 1 or 2 double bonds; alkynyl having 2 to 6 carbons and 1 or 2 triple bonds; aryl wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur; halo; C₁ to C₁₂ haloalkyl; hydroxyl; C₁ to C₁₂ alkoxy; C₃ to C₂₀ arylalkyloxy; C₁ to C₁₂ alkylcarbonyl; formyl; oxycarbonyl; carboxy; C₁ to C₁₂ alkyl carboxylate; C₁ to C₁₂ alkyl amide; aminocarbonyl; amino; cyano; diazo; nitro; thio; sulfoxyl; sulfonyl groups; or a group selected from the group consisting of

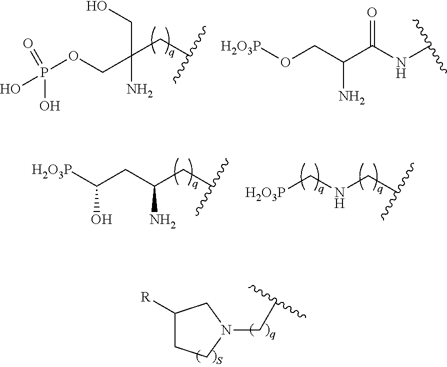

wherein R is CO₂H or PO₃H₂, p is an integer of 1 or 2 and q is 0 or an integer of 1 to 5 and s is 0 or an integer of 1 or 2; provided that, if Y is phenyl, it must be substituted with at least one R⁴ group that is not hydrogen.

Examples of such compounds include the following

| NO. | COMPOUND |
|---|---|
| 1 | 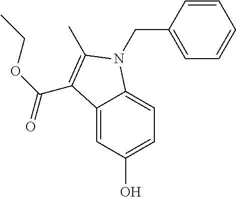 |
| 2 | 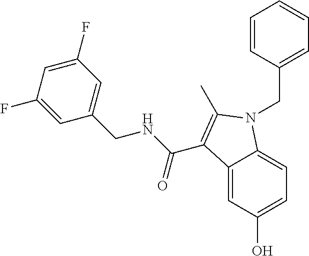 |
| 3 | 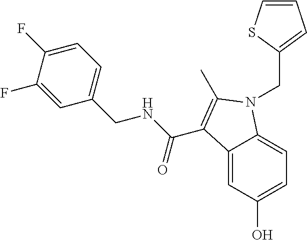 |
| 4 | 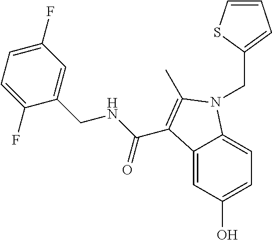 |
| 5 | 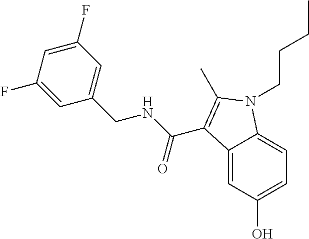 |
| 6 | 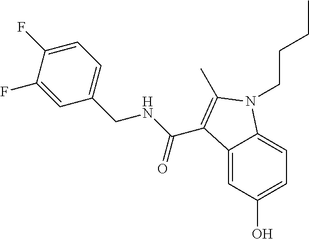 |
| 7 | 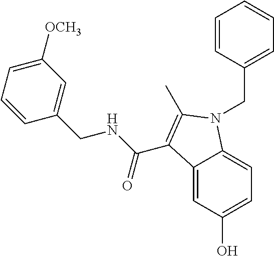 |
| 8 | 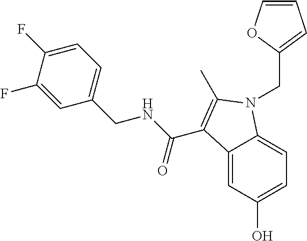 |
| 9 | 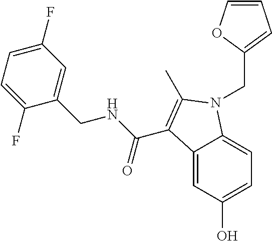 |
| 10 | 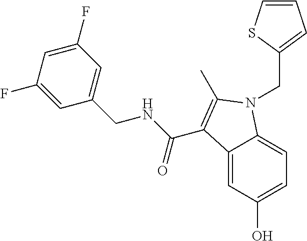 |
| 11 | 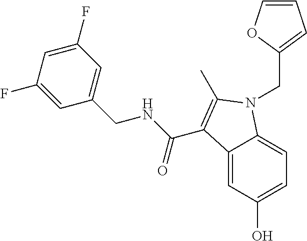 |
| 12 | 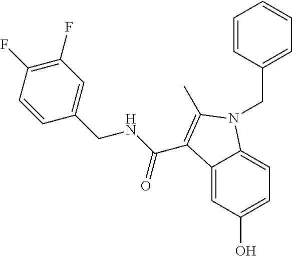 |
| 13 | 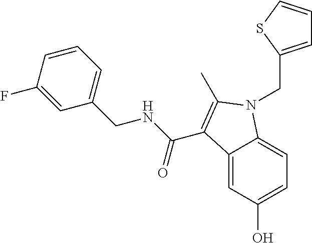 |
| 14 | 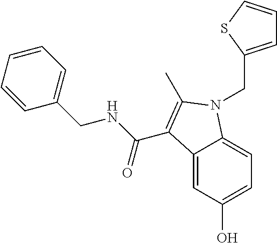 |
| 15 | 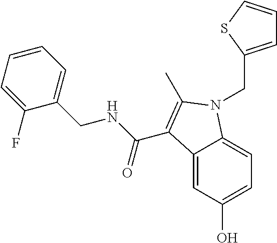 |
| 16 | 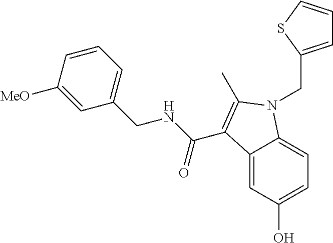 |
| 17 | 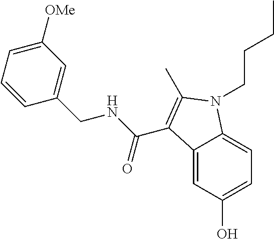 |
| 18 | 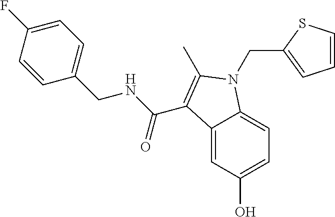 |
| 19 | 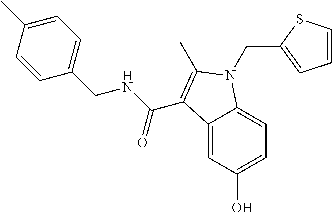 |
| 20 | 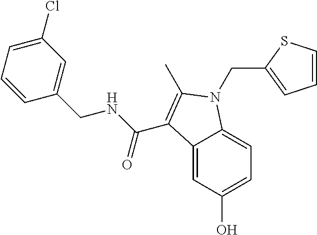 |
| 21 | 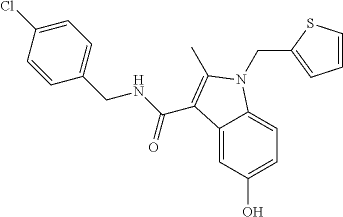 |
| 22 | 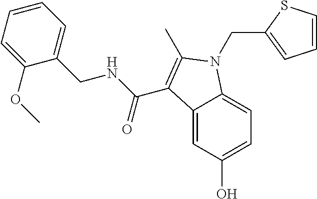 |
| 23 | 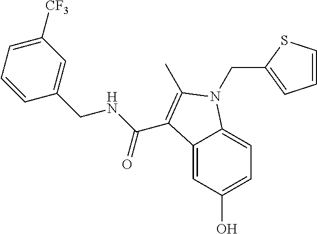 |
| 24 | 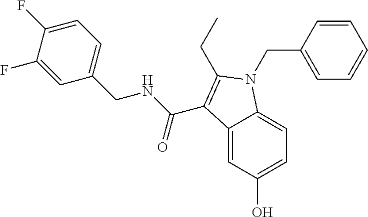 |
| 25 | 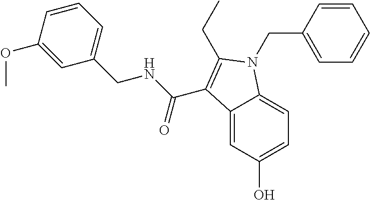 |
| 26 | 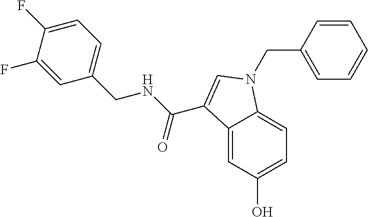 |
| 27 | 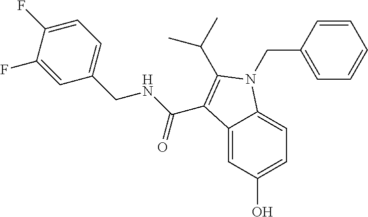 |
| 28 | 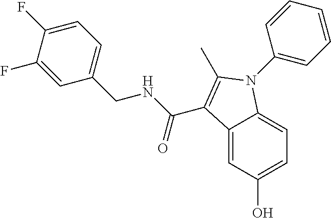 |
| 29 | 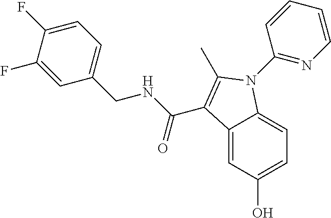 |
| 30 | 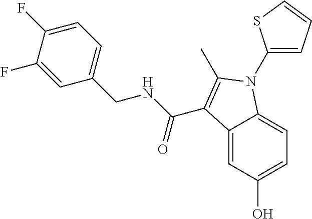 |
| 31 | 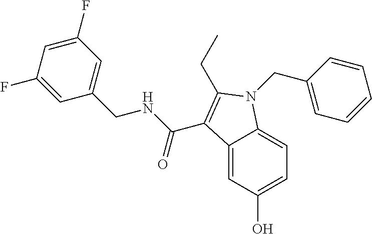 |
| 32 | 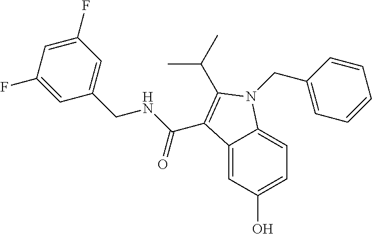 |
| 33 | 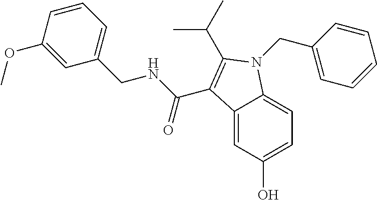 |
| 34 | 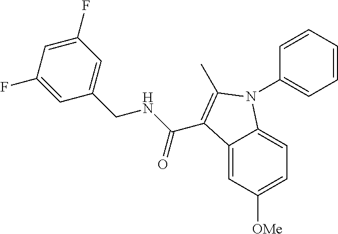 |
| 35 | 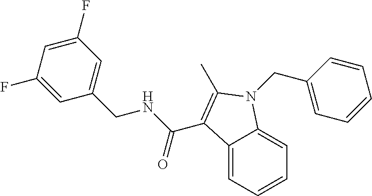 |
| 36 | 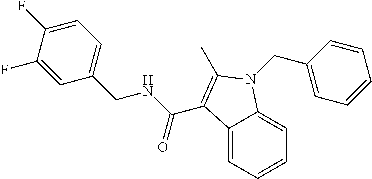 |
| 37 | 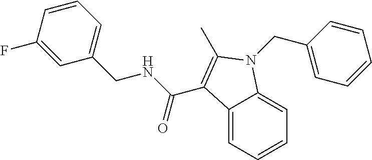 |
| 38 | 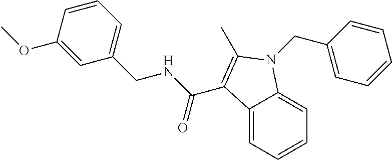 |
| 39 | 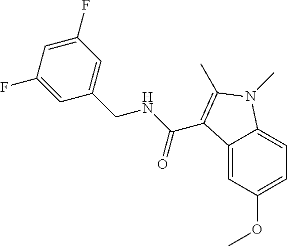 |
| 40 | 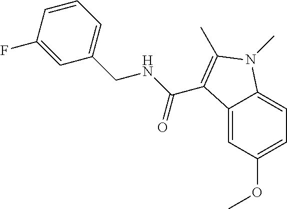 |
| 41 | 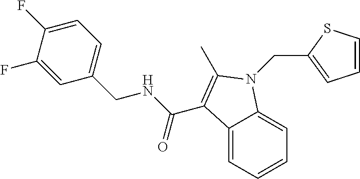 |
| 42 | 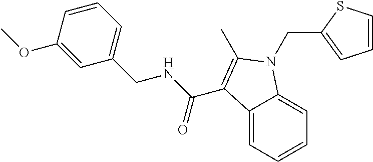 |
| 43 | 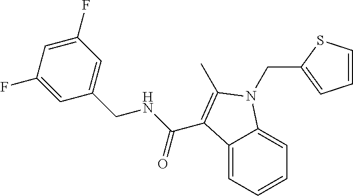 |
| 44 | 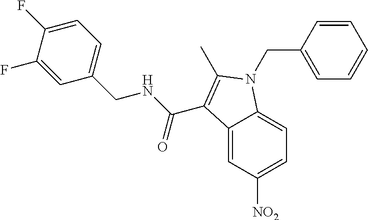 |
| 45 | 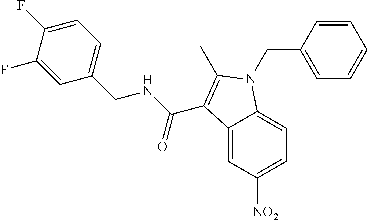 |
| 46 | 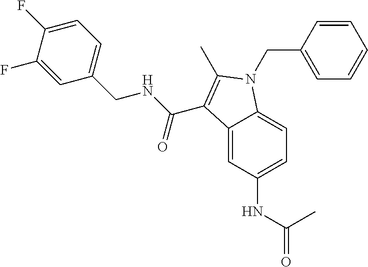 |
| 47 | 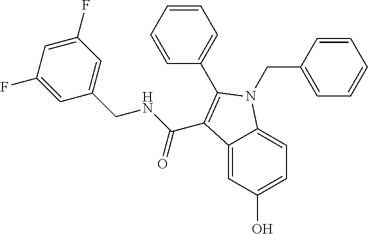 |
| 48 | 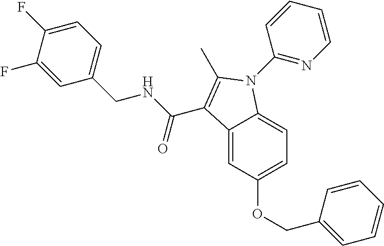 |

### Additional Indole Compounds

U.S. patent application Ser. No. 12/013,239 discloses S1P3 receptor antagonists having the following formula:

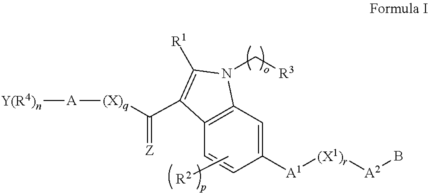

wherein:

R¹, R², R³ and R⁴ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, carbocyclic hydrocarbon groups having from 3 to 20 carbon atoms, heterocyclic groups having up to 20 carbon atoms and at least one of oxygen, nitrogen and/or sulfur in the ring, halo, C₁ to C₁₂ haloalkyl, hydroxyl, C₁ to C₁₂ alkoxy, C₃ to C₂₀ arylalkyloxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, and sulfonyl groups;

X and X¹ are independently selected from the group consisting of NR⁵, O and S;

R⁵ is hydrogen, an alkyl group of 1 to 10 carbons, a cycloalkyl group of 5 to 10 carbons, phenyl or lower alkylphenyl;

Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;

Z is O or S;

n is 0 or an integer of from 1 to 5;

o is 0 or an integer of from 1 to 3;

p is 0 or an integer of from 1 to 3;

q is 0 or 1;

r is 0 or 1;

A, A¹ and A² are independently selected from the group consisting of (CH₂)ᵥ wherein v is 0 or an integer of from 1 to 12, branched chain alkyl having 3 to 12 carbons, cycloalkyl having 3 to 12 carbons, alkenyl having 2 to 10 carbons and 1-3 double bonds and alkynyl having 2 to 10 carbons and 1 to 3 triple bonds;

B is selected from the group consisting of hydrogen, OR⁶, COOR⁷, NR⁸R⁹, CONR⁸R⁹, COR¹⁰, CH═NOR¹¹, CH═NNR¹²R¹³ wherein R⁶, R⁷, R¹⁰ and R¹¹ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, R⁸, R⁹, R¹² and R¹³ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, or R⁸ and R⁹ and/or R¹² and R¹³, together, can form a divalent carbon radical of 2 to 5 carbons to form a heterocyclic ring with nitrogen, wherein any of R⁶, R⁷, R⁸, R⁹, R¹⁰, R¹¹, R¹² of R¹³ may be substituted with one or more halogen, hydroxy, alkyloxy, cyano, nitro, mercapto or thiol radical; provided however, when v is 0, and r is 0, B is not hydrogen; or B is a carbocyclic hydrocarbon group having from 3 to 20 carbon atoms, or a heterocyclic group having up to 20 carbon atoms and at least one of oxygen, nitrogen and/or sulfur in the ring, and wherein when said B is a carbocyclic or heterocyclic group B may be bonded to A² at any position, or a pharmaceutically acceptable salt of said compound.

The aryl group is a carbocyclic aryl or heterocyclic aryl group wherein said carbocylic aryl comprises from 6 to 20 atoms and said heterocyclic aryl comprise from 2 to 20 carbon atoms and from 1 to 5 heteroatoms selected from the group consisting of nitrogen, oxygen and sulfur, and preferably said aryl group is selected from the group consisting of benzene, pyridine, pyrazine, pyridazine, pyrimidine, triazine, thiophene, furan, thiazole, thiadiazole, isothiazole, oxazole, oxadiazole, isooxazole, naphthalene, quinoline, tetralin, chroman, thiochroman, tetrahydroquinoline, dihydronaphthalene, tetrahydronaphthalen, chromene, thiochromene, dihydroquinoline, indan, dihydrobenzofuran, dihydrobenzothiophene, indene, benzofuran, benzothiophene, coumarin and coumarinone. Said aryl groups can be bonded to the above moiety at any position. Said aryl group may itself be substituted with any common organic functional group including but not limited to C₁ to C₁₂ alkyl, C₂ to C₆ alkenyl, C₂ to C₆ alkynyl, halo, C₁ to C₁₂ haloalkyl, hydroxyl, C₁ to C₁₂ alkoxyl, C₁ to C₁₂ alkylcarbonyl, formyl, oxycarbonyl, carboxyl, C₁ to C₁₂ alkyl carboxylate, C₁ to C₁₂ alkyl amide, aminocarbonyl, amino, cyano, diazo, nitro, thio, sulfoxyl, or sulfonyl groups.

Preferably Z is O.

Preferably, the carbocyclic aryl group will comprise from 6 to 14 carbon atoms, e.g. from 6 to 10 carbon atoms. Preferably the heterocyclic aryl group will comprise from 2 to 14 carbon atoms and one or more, e.g. from 1 to 3 heteroatoms selected from the group consisting of nitrogen, oxygen and sulfur.

Preferably, A is CH₂.

Preferably, X is NH.

Preferably, n is 0 or an integer of 1 or 2 and R⁴ is fluoro.

Preferably, R¹ is i-propyl.

Preferably, R³ is selected from the group consisting of phenyl, which may be substituted with one or two fluoro groups, and pyridyl.

Preferably, p is 0.

Preferably, A¹ and A² are absent.

Preferably, B is OR⁶ or COOR⁷.

Preferably, X is O, r is 1, A¹ is absent, A² is (CH₂)ᵥ, wherein v is 1 or 2, and B is OR⁶ or NR⁸R⁹, and R⁶, R⁸ and R⁹ are methyl.

Preferably, B is CR¹⁰═NOR¹¹R¹⁰ wherein R¹⁰ is H and R¹¹ is methyl or i-butyl or B is CONR⁸R⁹ wherein R⁸ and R⁹ are selected from the group consisting of H, methyl, ethyl and propyl, or R⁸ and R⁹, together with N, form a 5-member ring.

Preferably, A¹ is absent, r is 0, A² is CH₂ and B is OR⁶, wherein R⁶ is H, or X is O, r is 1 and B is COR¹⁰, wherein R¹⁰ is methyl.

Examples of such compounds include the following:

| No. | Compound |
|---|---|
| 49 | 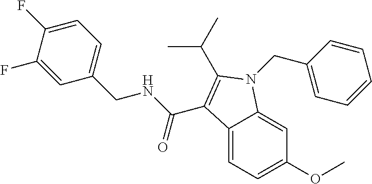 |
| 50 | 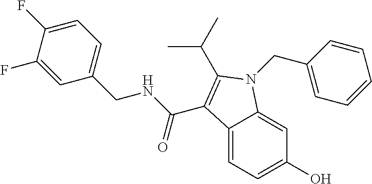 |
| 51 | 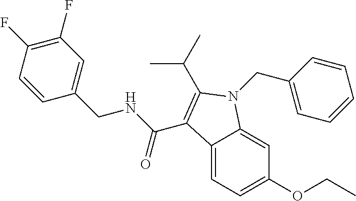 |
| 52 | 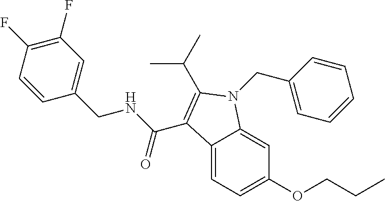 |
| 53 | 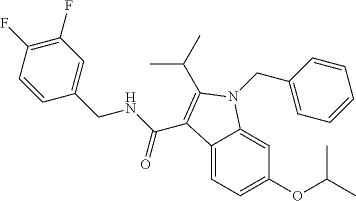 |
| 54 | 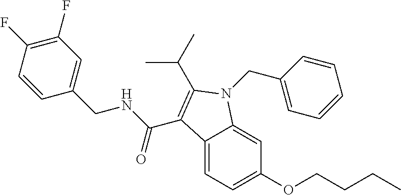 |
| 55 | 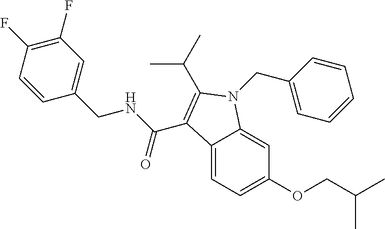 |
| 56 | 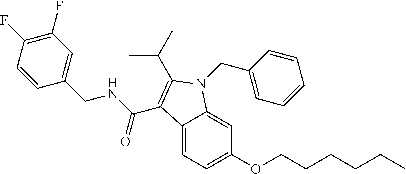 |
| 57 | 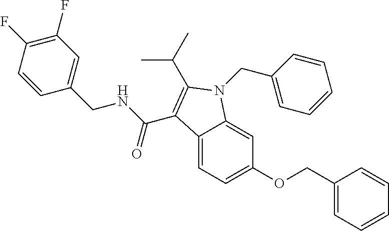 |
| 58 | 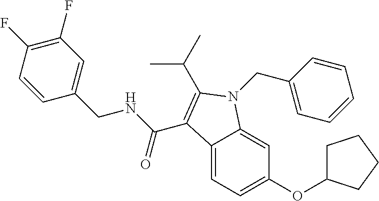 |
| 59 | 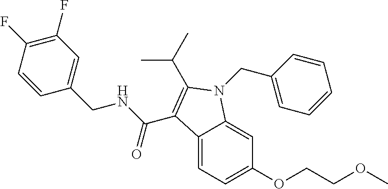 |
| 60 | 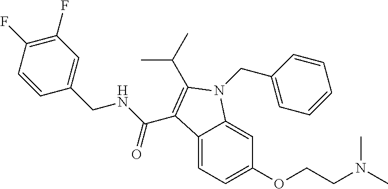 |
| 61 | 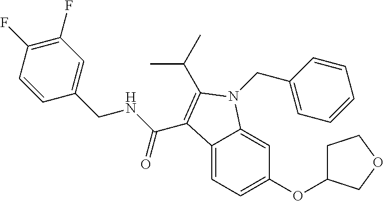 |
| 62 | 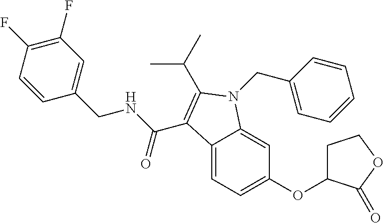 |
| 63 | 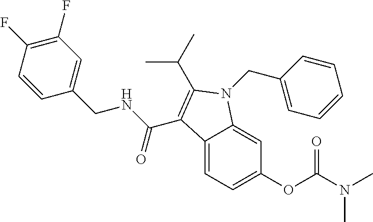 |
| 64 | 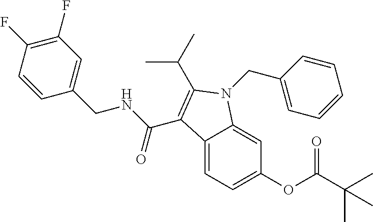 |
| 65 | 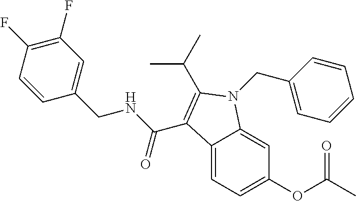 |
| 66 | 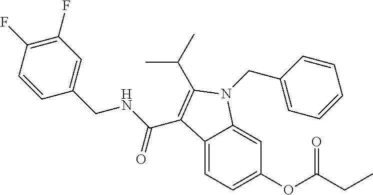 |
| 67 | 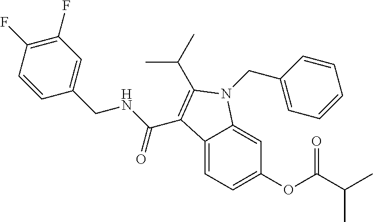 |
| 68 | 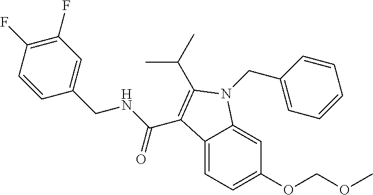 |
| 69 | 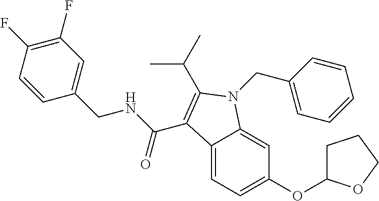 |
| 70 | 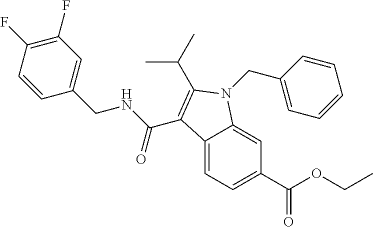 |
| 71 | 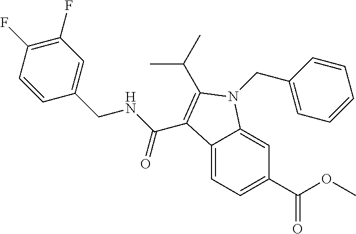 |
| 72 | 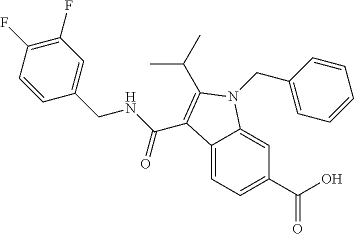 |
| 73 | 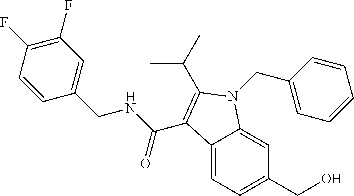 |
| 74 | 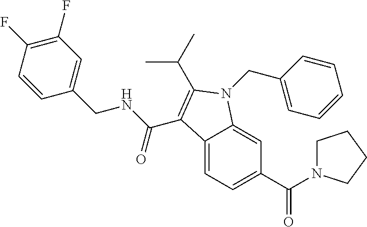 |
| 75 | 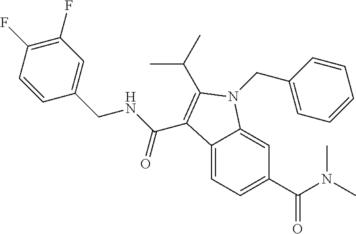 |
| 76 | 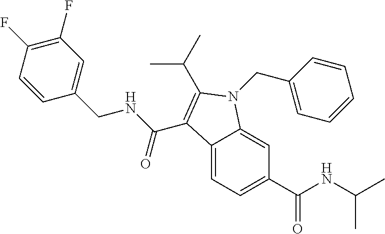 |
| 77 | 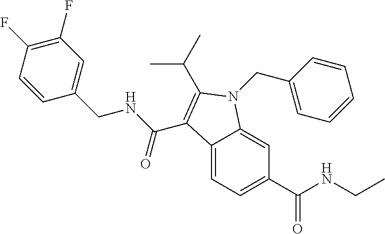 |
| 78 | 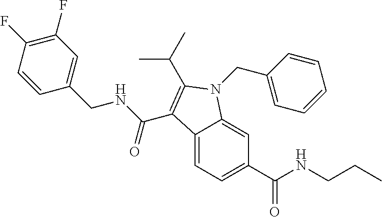 |
| 79 | 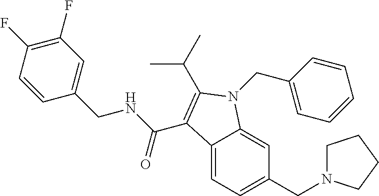 |
| 80 | 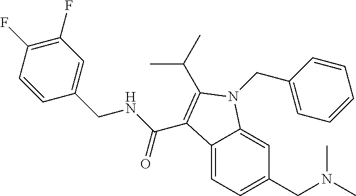 |
| 81 | 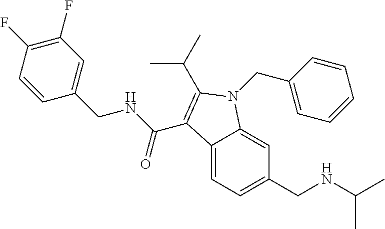 |
| 82 | 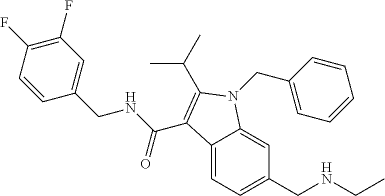 |
| 83 | 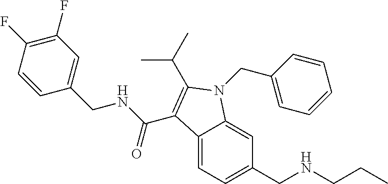 |
| 84 | 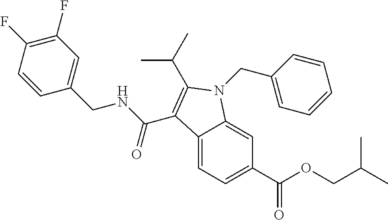 |
| 85 | 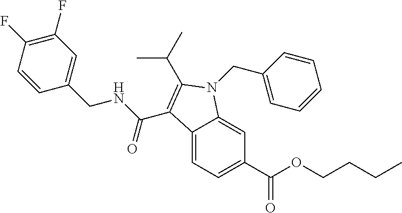 |
| 86 | 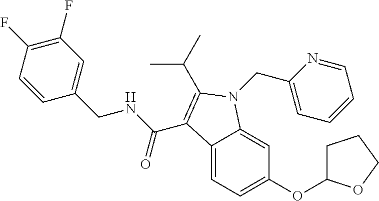 |
| 87 | 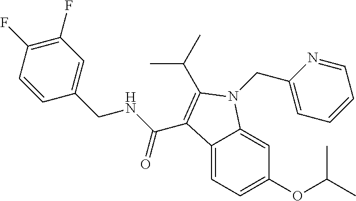 |
| 88 | 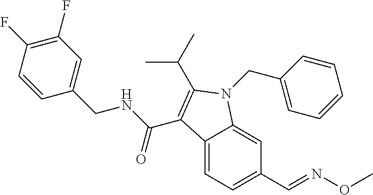 |
| 89 | 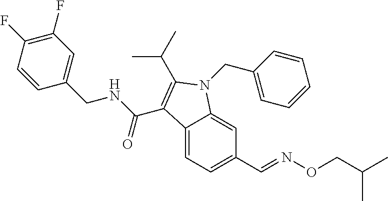 |
| 90 | 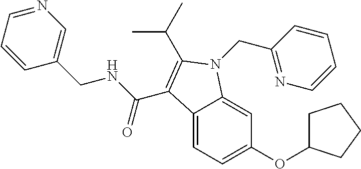 |
| 91 | 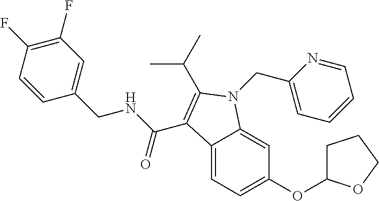 |
| 92 | 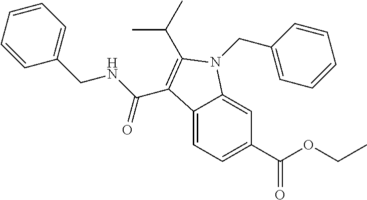 |
| 93 | 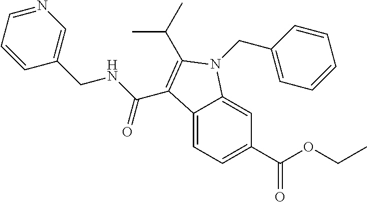 |
| 94 | 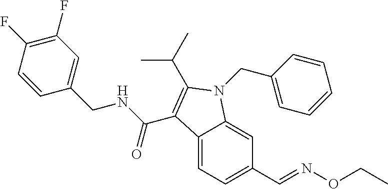 |
| 95 | 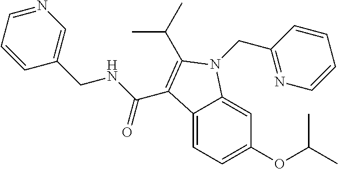 |
| 96 | 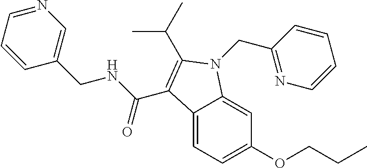 |
| 97 | 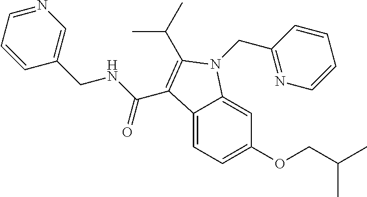 |
| 98 | 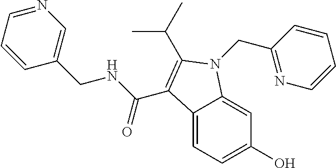 |
| 99 | 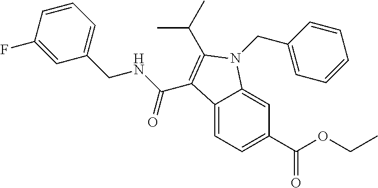 |
| 100 | 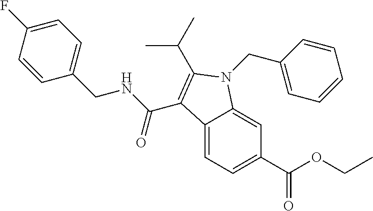 |
| 101 | 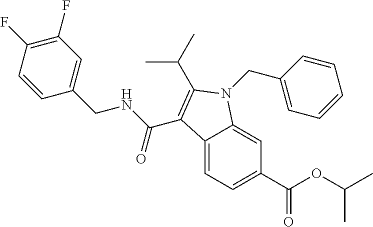 |
| 102 | 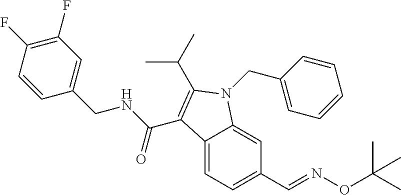 |
| 103 | 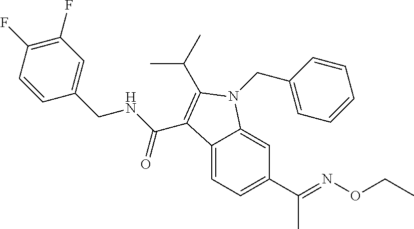 |
| 104 | 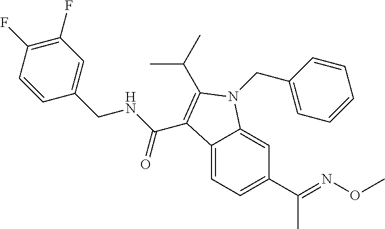 |
| 105 | 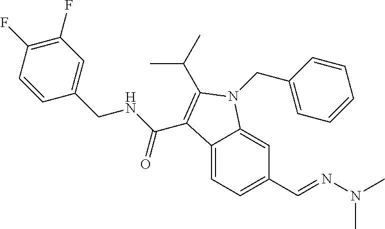 |
| 106 | 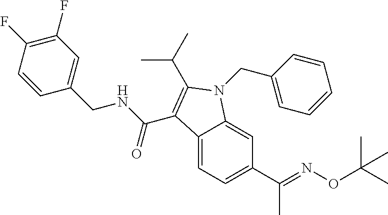 |
| 107 | 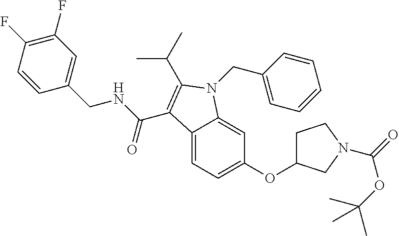 |
| 108 | 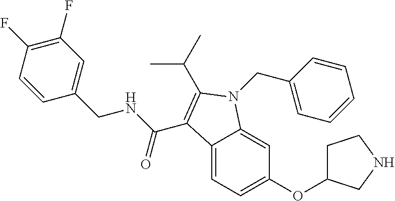 |
| 109 | 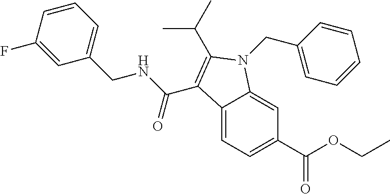 |
| 110 | 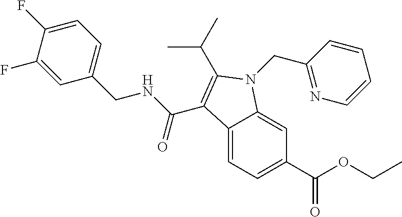 |
| 111 | 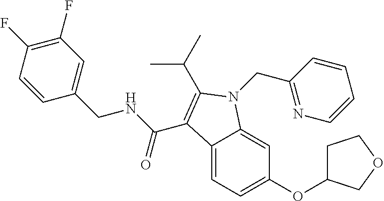 |
| 112 | 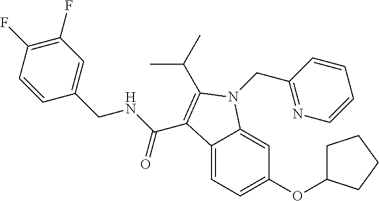 |
| 113 | 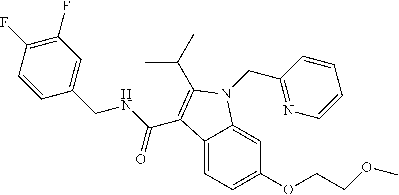 |
| 114 | 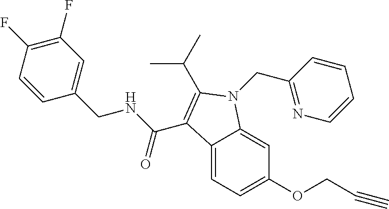 |
| 115 | 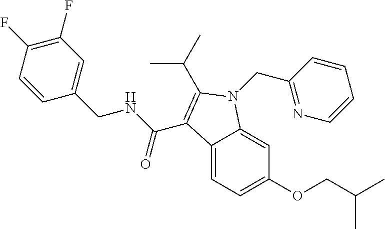 |
| 116 | 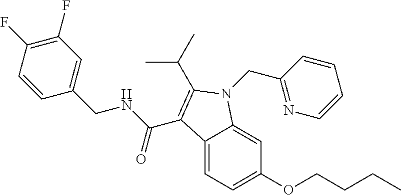 |
| 117 | 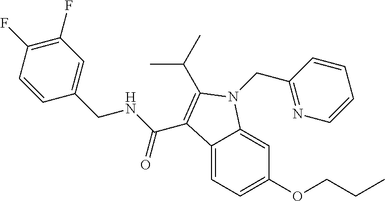 |
| 118 | 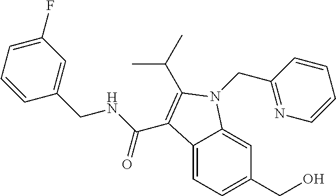 |
| 119 | 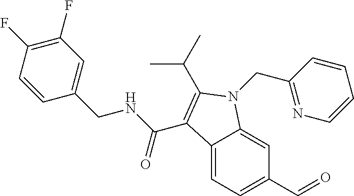 |
| 120 | 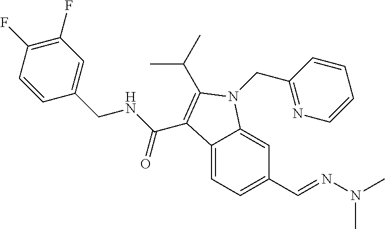 |
| 121 | 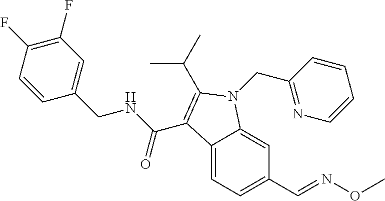 |

### Other Indole Compounds

Other compositions useful in the methods of the invention include those disclosed in U.S. patent application Ser. No. 11/690,637. That application discloses S1P3 receptor antagonists having the following formula:

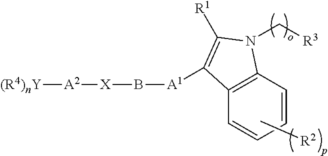

wherein:

A¹ and A² are independently selected from the group consisting of (CH₂)m where m is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

B is selected from the group consisting of (CH₂)n, where n is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, C═C(R⁵)₂, C═O, C═S, R⁵C═NR⁵, R⁵C═CR⁵, C═NOR⁵, CR⁵OR⁵, C(OR⁵)₂, CR⁵N(R⁵)₂, C(N(R⁵)₂)₂, CR⁵SR⁵, C(SR⁵)₂, SO, SO₂, and heterocyclic aryl comprising from 2 to 14 carbon atoms and from 1 to 3 heteroatoms selected from the group consisting of nitrogen, oxygen and sulfur;

X is selected from the group consisting of (CH₂)r, where r is O or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;

provided that when m is 0 and B is C═O then X is not NR⁵, O or S;

Y is R⁶, or a carbocyclic aryl group comprising from 6 to 14 carbon atoms or a heterocyclic aryl group comprising from 2 to 14 carbon atoms and from 1 to 3 heteroatoms selected from the group consisting of nitrogen, oxygen and sulfur;

o is 0 or an integer of from 1 to 3;

p is 0 or an integer of from 1 to 4;

R¹, R², R³, R⁴ are independently selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxy, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, sulfonyl ,

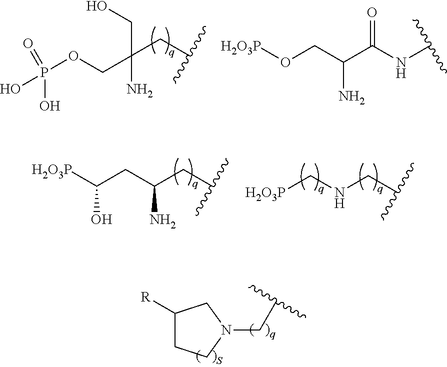

wherein R is CO₂H or PO₃H₂ and q is 0 or an integer of 1 to 5 and s is 0 or an integer from 1 to 3;

R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl; and

R⁶ is selected from the group consisting of straight or branched chain alkyl having 1 to 12 carbons, cycloalkyl having 3 to 6 carbons, alkenyl having 2 to 6 carbons and 1 or 2 double bonds and alkynyl having 2 to 6 carbons and 1 or 2 triple bonds.

Examples of such compounds include the following.

| No. | COMPOUND |
|---|---|
| 122 | 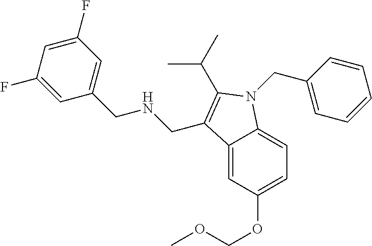 |
| 123 | 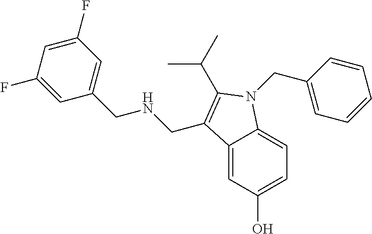 |
| 124 | 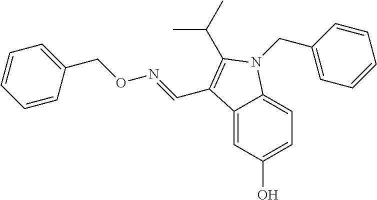 |
| 125 | 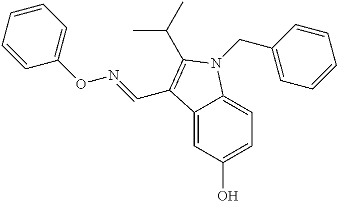 |
| 126 | 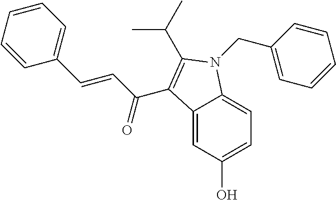 |
| 127 | 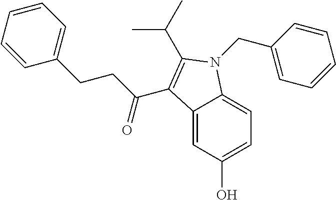 |
| 128 | 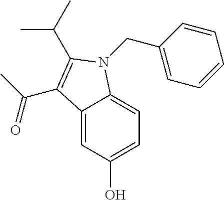 |
| 129 | 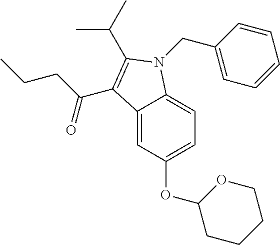 |
| 130 | 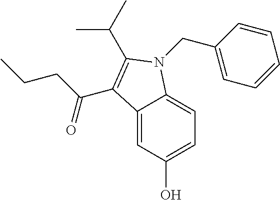 |
| 131 | 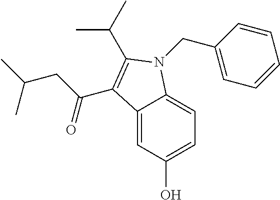 |
| 132 | 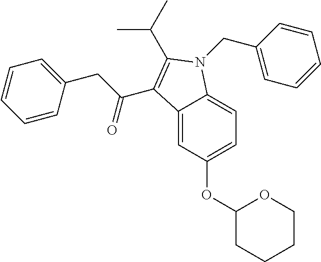 |
| 133 | 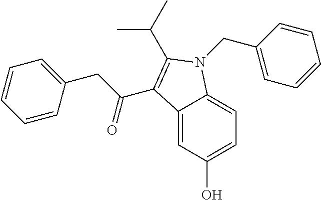 |
| 134 | 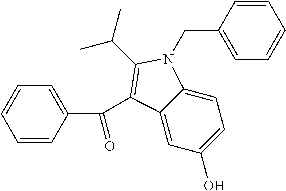 |
| 135 | 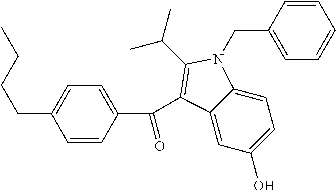 |
| 136 | 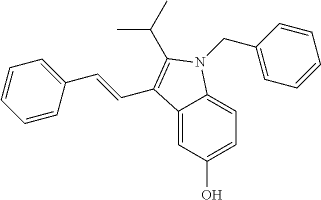 |
| 137 | 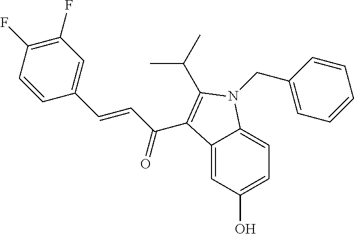 |
| 138 | 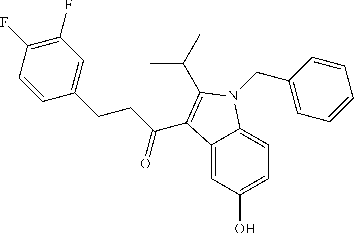 |
| 139 | 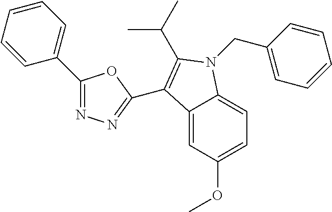 |
| 140 | 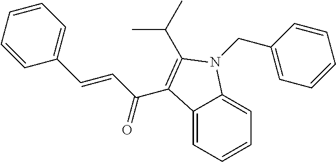 |
| 141 | 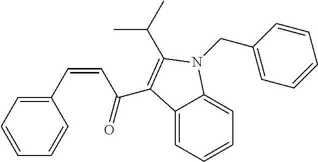 |
| 142 | 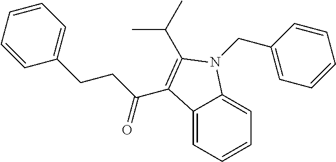 |
| 143 | 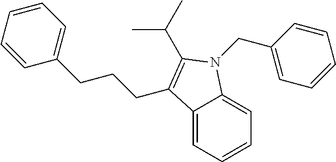 |
| 144 | 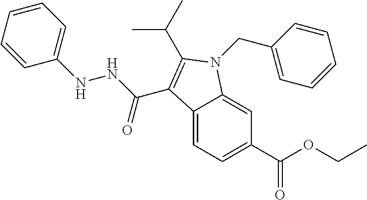 |
| 145 | 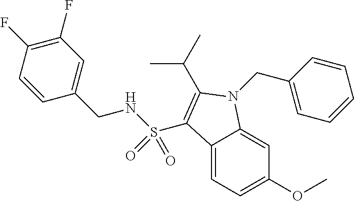 |

### Heteraromatic Compounds

Other compositions useful in the methods of the invention include those disclosed in U.S. patent application Ser. No. 11/850,756. That application discloses S1P3 receptor antagonists having the following formula:

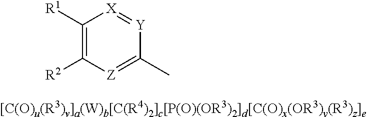

wherein

X is selected from the group consisting of CR³ and N;

Y is selected from the group consisting of CR³ and N;

Z is selected from the group consisting of CR³ and N;

at least one of X, Y and Z is N;

W is NR³ or O;

R¹ is an aryl group;

R² is an aryl group;

R³ is selected from the group consisting of H and alkyl; and 2 of said R³ groups may together with N may form a heterocylic ring having from 2 to 6 carbon atoms; R⁴ is selected from the group consisting of H, alkyl, OR³, and N(R³)₂;

a is 0 or an integer of from 1 to 6;

b is 0 or 1;

c is 0 or an integer of from 1 to 6;

d is 0 or 1;

e is 0 or 1;

u is 0 or 1;

v is 0 or an integer of from 1 to 2;

x is 0 or 1;

y is 0 or an integer of from 1 to 3;

z is 0 or an integer of from 1 to 3;

provided, however, that when d is 0, e is 1, and when e is 0, d is 1.

Examples of such compounds include the following. Several of these selectively inhibit the S1P3 receptor subtype as compared to at least the S1P1 receptor subtypes. The EC₅₀ and IC₅₀ values expressed in the following table were obtained in the FLIPR assay described above. EC₅₀ or IC₅₀ values are stated first, followed by percent efficacy or percent inhibition stated in parenthesis. In this table and the next, percent efficacy is defined as percent of receptor activity induced by a test compound at the highest dose tested (10 μM) relative to the receptor activity induced by 5 nM sphingosine-1-phosphate, and percent inhibition is defined as percent of receptor activity induced by 5 nM sphingosine-1-phosphate that is inhibited by a test compound at the highest dose tested (10 μM). “NA” means that no activity was detected at highest dose tested; “ND” means not determined.

| | | S1P1 | S1P3 |
|---|---|---|---|
| No. | COMPOUND | (EC50) | (IC50) |
| 146 | 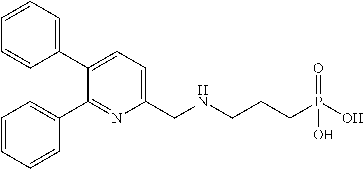 | ND | 1.6 μM (83) |
| 147 | 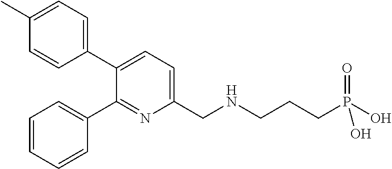 | 121 nM (36) | 231 nM (98) |
| 148 | 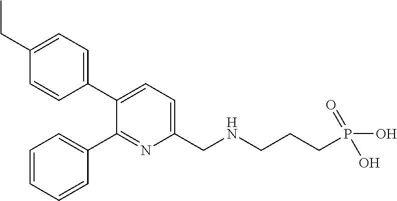 | 170 nM (57) | 319 nM (98) |
| 149 | 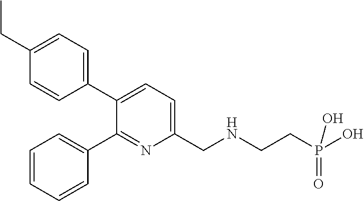 | NA | 1.8 μM (99) |
| 150 | 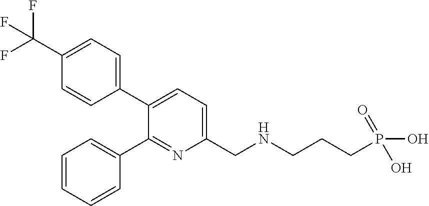 | ND | ND (95) |
| 151 | 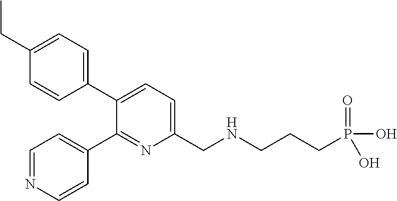 | NA | 1.1 μM (95) |
| 152 | 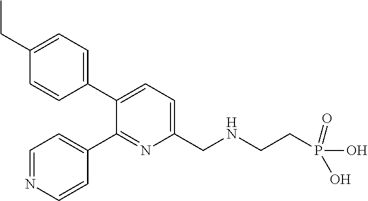 | NA | 1.8 μM (68) |
| 153 | 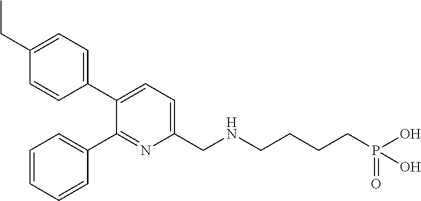 | NA | ND (30) |
| 154 | 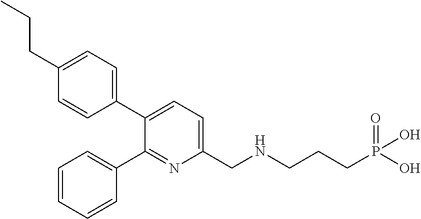 | 114 nM (69) | 319 nM (98) |
| 155 | 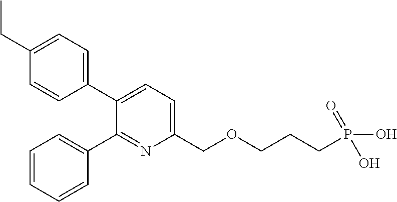 | NA | 4.0 μM (27) |
| 156 | 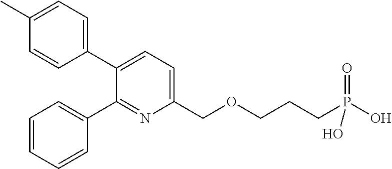 | NA | 1.9 μM (11) |
| 157 | 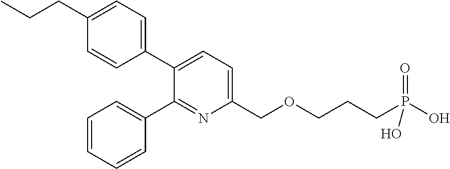 | NA | ND |

### Additional Selective S1P3 Receptor Inhibitors

Examples of compounds that selectively inhibit the S1P3 receptor subtype as compared to at least the S1P1 and S1P2 receptor subtypes include the following. The IC₅₀ values expressed below were obtained in the FLIPR assay described above. IC₅₀ values are stated first (except as otherwise noted), followed by percent efficacy or percent inhibition in parenthesis.

| | S1P1 | S1P2 | S1P3 |
|---|---|---|---|
| STRUCTURE | (IC₅₀) | (IC₅₀) | (IC₅₀) |
| 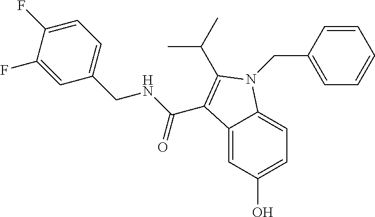 | NA | NA | 35 nM (98) |
| 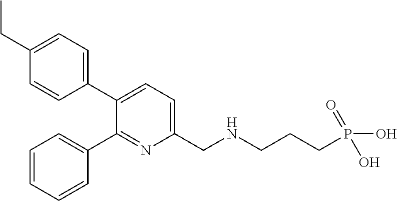 | EC₅₀ = 170 nM (57) | NA | 319 nM (98) |
| 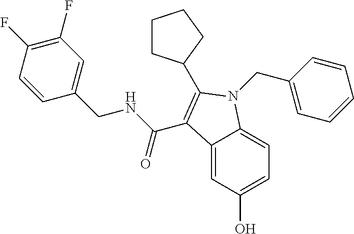 | NA | NA | 31 nM (100) |
| 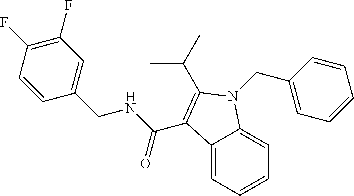 | NA | NA | 209 nM (100) |
| 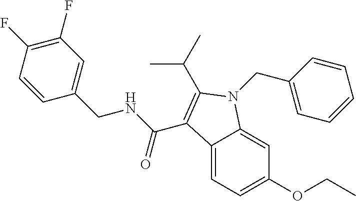 | NA | NA | 19 nM (100) |
| 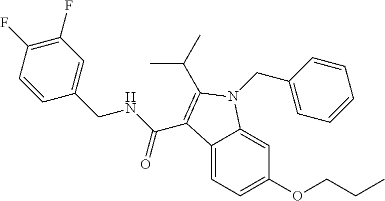 | NA | NA | 5 nM (100) |
| 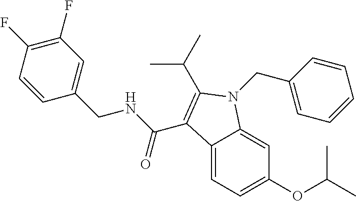 | NA | NA | 6 nM (100) |
| 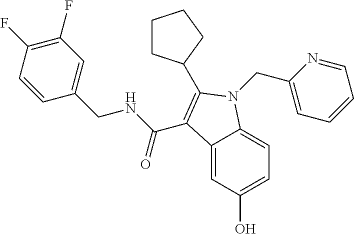 | NA | NA | 17 nM (99) |

### S1P3 Inverse Agonists

U.S. Patent Publication No. 2005/022422 discloses S13P receptor inhibitors that are inverse agonists of S1P3. The inhibitors have the following formula

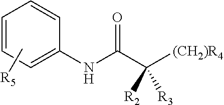

wherein R₂ is H, R₃ is NH₂, R₄ is phosphate, and R₅ is (CH₂)₇CH₃, wherein R₅ may be in the ortho or meta position.

### Thiazolidine S1P3 Antagonists

U.S. Patent Application Publication No. 2008/0025973 (the “'973 publication”) discloses S1P3 receptor inhibitors having the following structures:

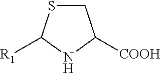

wherein R₁ is C₆-C₁₃ alkyl, or alkyl-substituted aryl where the substitution is C₅-C₉ alkyl;

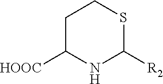

wherein where R₂ is C₉-C₁₃ alkyl; and

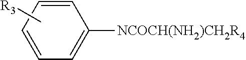

wherein R₃ is o- or m-C₅-C₈ alkyl; and R₄ is phosphate, phosphate analog, phosphonate, or sulfate. As used here, “phosphate analog” includes phosphoro-thioates, -dithioates, -selenoates, -diselenoates, -anilothioates, -anilidates, -amidates, and boron phosphates, for example.

### Pharmaceutically Acceptable Salts

One can use in the compositions and methods of the invention any S13P receptor inhibitor as its pharmaceutically acceptable salt.

A “pharmaceutically acceptable salt” is any salt which retains the biological effectiveness and properties of the free bases and which are obtained by reaction with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. A pharmaceutically acceptable salt also refers to any salt which may form in vivo as a result of administration of an acid, another salt, or a prodrug which is converted into an acid or salt.

Pharmaceutically acceptable salts of acidic functional groups may be derived from organic or inorganic bases. The salt may comprise a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

### Prodrugs

One can use in the methods of the invention a prodrug of any of the compositions of the invention.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of C1 (i.e., the terminal carboxylic acid of a natural prostaglandin), or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

The S1P3 receptor inhibitors of the invention may be either synthetically produced, or may be produced within the body after administration of a prodrug. Hence, “S1P3 receptor inhibitor” encompasses compounds produced by a manufacturing process and those compounds formed in vivo only when another drug administered.

### Isomers and Racemates

One can use in the compositions and methods of the invention an enantiomer, stereoisomer, or other isomer of any S13P receptor inhibitor.

### Inflammation

Inflammation refers to any enlargement of organs or tissues caused by an immune response to injury or antigens. The injury may be mechanical (e.g., an incision made by a scalpel, a contusion caused by a fall), chemical (e.g., irritation to lung tissue from inhaling smoke), or biological (e.g., injury to vascular tissue from free radicals produced by the oxidation of polyunsaturated fats). Antigens may be presented by bacteria, viruses, and other pathogens, or may be presented by a patient's own tissue, as is the case with autoimmune disorders. An immune response to an Injury or antigen causes blood flow and vascular permeability to increase, allowing chemotactic peptides, neutrophils, mononuclear cells, and other immune system components to leave the intravascular compartment, leading to enlargement of the space surrounding site of vascular permeability. The methods of the invention may be used to treat any such enlargement.

“Inflammation,” as used here, includes conditions in which inflammation is the primary component of a disease (e.g., bursitis and tendonitis) and also those conditions in which inflammation is not the primary component (e.g., sepsis)).

The compounds of the invention may be used to treat inflammation of any tissue and organs of the body, including musculoskeletal inflammation, vascular inflammation, neural inflammation, digestive system inflammation, ocular inflammation, inflammation of the reproductive system, and other inflammation, as exemplified below.

Musculoskeletal inflammation refers to any inflammatory condition of the musculoskeletal system, particularly those conditions affecting skeletal joints, including joints of the hand, wrist, elbow, shoulder, jaw, spine, neck, hip, knee, ankle, and foot, and conditions affecting tissues connecting muscles to bones, such as tendons. Examples of musculoskeletal inflammation which may be treated with compounds of the invention include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystica).

Ocular inflammation refers to inflammation of any structure of the eye, including the eye lids. Examples of ocular inflammation which may be treated with the compounds of the invention include blepharitis, blepharochalasis, conjunctivitis, dacryoadenitis, keratitis, keratoconjunctivitis sicca (dry eye), scleritis, trichiasis, and uveitis.

Examples of inflammation of the nervous system which may be treated with the compounds of the invention include encephalitis, Guillain-Barre syndrome, meningitis, neuromyotonia, narcolepsy, multiple sclerosis, myelitis, and schizophrenia.

Examples of inflammation of the vasculature or lymphatic system which may be treated with the compounds of the invention include arthrosclerosis, arteritis, phlebtiis, vasculitis, and lymphangitis.

Examples of inflammatory conditions of the digestive system which may be treated with the compounds of the invention include cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system which may be treated with the compounds of the invention include cervicitis, chorioamnionitis, endometritis, epididymitis, omphalitis, oophoritis, orchitis, salpingitis, tubo-ovarian abscess, urethritis, vaginitis, vulvitis, and vulvodynia.

The compounds of the invention may be used to treat autoimmune conditions having an inflammatory component. Such conditions include acute disseminated alopecia universalis, Behçet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, crohn's disease, diabetes mellitus type 1, goodpasture's syndrome, Guillain-Barré syndrome, Hashimoto's disease, Kawasaki's disease, lupus erythematosus, mixed connective tissue disease, multiple sclerosis, opsoclonus myoclonus syndrome, optic neuritis, ord's thyroiditis, pemphigus, rheumatoid arthritis, Reiter's syndrome, Sjögren's syndrome, temporal arteritis, Wegener's granulomatosis, warm autoimmune hemolytic anemia, interstitial cystitis, lyme disease, morphea, psoriasis, sarcoidosis, scleroderma, ulcerative colitis, and vitiligo.

The compounds of the invention may be used to treat T-cell mediated hypersensitivity diseases having an inflammatory component. Such conditions include contact hypersensitivity, delayed-type hypersensitivity, contact dermatitis (including that due to poison ivy), uticaria, skin allergies, respiratory allergies (hayfever, allergic rhinitis) and gluten-sensitive enteropathy (Celiac disease);

Other inflammatory conditions which may be treated with the compounds of the invention include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatitis, transplant rejection (involving organs such as kidney, liver, heart, lung, pancreas (e.g., islet cells), bone marrow, cornea, small bowel, skin allografts, skin homografts, and heart valve xenografts, serum sickness, and graft vs. host disease), acute pancreatitis, chronic pancreatitis, acute respiratory distress syndrome, Sezary's syndrome, congenital adrenal hyperplasia, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and iridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) hemolytic anemia, leukemias and lymphomas in adults, acute leukemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosis, psoriasis, chronic pulmonary disease, and inflammation accompanying infectious conditions (e.g., sepsis).

### Administration

One can use any of the compounds described above to treat inflammation. To “treat,” as used here, means to deal with medically. It includes both preventing inflammation and relieving it, whether such prevention or relief is complete or partial.

### Dose

The precise dose and frequency of administration depends on the severity and nature of the patient's condition, on the manner of administration, on the potency and pharmacodynamics of the particular compound employed, and on the judgment of the prescribing physician. Determining dose is a routine matter that is well within the capability of someone of ordinary skill in the art.

The compositions of the invention may be administered orally or parenterally, the later by subcutaneous injection, intramuscular injection, intravenous administration, or other route.

### Excipients and Dosage Forms

Those skilled in the art will readily understand that for administering pharmaceutical compositions of the invention the S13P receptor inhibitor may be admixed with pharmaceutically acceptable excipient which are well known in the art.

A pharmaceutical composition to be administered systemically may be confected as a powder, pill, tablet or the like, or as a solution, emulsion, suspension, aerosol, syrup or elixir suitable for oral or parenteral administration or inhalation.

For solid dosage forms or medicaments, non-toxic solid carriers include, but are not limited to, pharmaceutical grades of mannitol, lactose, starch, magnesium stearate, sodium saccharin, the polyalkylene glycols, talcum, cellulose, glucose, sucrose and magnesium carbonate. The solid dosage forms may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in U.S. Pat. No. 4,256,108, No. 4,166,452, and No. 4,265,874 to form osmotic therapeutic tablets for control release. Liquid pharmaceutically administrable dosage forms can, for example, comprise a solution or suspension of one or more of the presently useful compounds and optional pharmaceutical adjutants in a carrier, such as for example, water, saline, aqueous dextrose, glycerol, ethanol and the like, to thereby form a solution or suspension. If desired, the pharmaceutical composition to be administered may also contain minor amounts of nontoxic auxiliary substances such as wetting or emulsifying agents, pH buffering agents and the like. Typical examples of such auxiliary agents are sodium acetate, sorbitan monolaurate, triethanolamine, sodium acetate, triethanolamine oleate, etc. Actual methods of preparing such dosage forms are known, or will be apparent, to those skilled in this art; for example, see Remington's Pharmaceutical Sciences, Mack Publishing Company, Easton, Pa., 16th Edition, 1980. The composition of the formulation to be administered, in any event, contains a quantity of one or more of the presently useful compounds in an amount effective to provide the desired therapeutic effect.

Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol and the like. In addition, if desired, the injectable pharmaceutical compositions to be administered may also contain minor amounts of non-toxic auxiliary substances such as wetting or emulsifying agents, pH buffering agents and the like.

1.-2. (canceled)

3. A method for treating inflammation, the method comprising the step of administering to a patient in need of such treatment a compound represented by the general formula 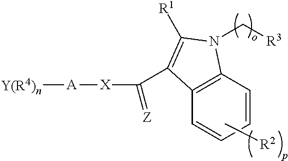
wherein
X is NR⁵, O, S;
Z is O or S;
n is 0 or an integer of from 1 to 4;
o is 0 or an integer of from 1 to 3;
p is 0 or an integer of from 1 to 4;
A is (C(R⁵)₂)m, wherein
m is 0 or an integer of from 1 to 6;
R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl groups;
Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;
R¹, R², R³, R⁴ are selected from the group consisting of hydrogen; straight or branched chain alkyl having 1 to 12 carbons; cycloalkyl having 3 to 6 carbons; alkenyl having 2 to 6 carbons and 1 or 2 double bonds; alkynyl having 2 to 6 carbons and 1 or 2 triple bonds; aryl wherein said aryl is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur; halo; C₁ to C₁₂ haloalkyl; hydroxyl; C₁ to C₁₂ alkoxy; C₃ to C₂₀ arylalkyloxy; C₁ to C₁₂ alkylcarbonyl; formyl; oxycarbonyl; carboxy; C₁ to C₁₂ alkyl carboxylate; C₁ to C₁₂ alkyl amide; aminocarbonyl; amino; cyano; diazo; nitro; thio; sulfoxyl; sulfonyl groups; or a group selected from the group consisting of 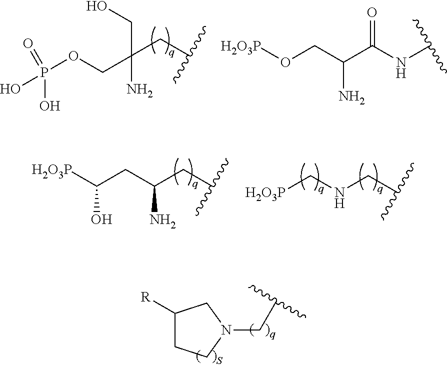
wherein R is CO₂H or PO₃H₂, p is an integer of 1 or 2 and q is 0 or an integer of 1 to 5 and s is 0 or an integer of 1 or 2; provided that, if Y is phenyl, it must be substituted with at least one R⁴ group that is not hydrogen.

4. The method of claim 3 wherein Z is O.

5. The method of claim 3 wherein Y is a phenyl group, or a heterocyclic aryl group selected from the group consisting of pyridyl, thienyl, furyl, pyradizinyl, pyrimidinyl, pyrazinyl, thiazolyl, oxazolyl, and imidazolyl.

6. The method of claim 5 wherein each said aryl is independently selected from the group consisting of phenyl, pyridine, pyrazine, pyridazine, pyrimidine, triazine, thiophene, furan, thiazole, thiadiazole, isothiazole, oxazole, oxadiazole, isooxazole, naphthalene, quinoline, tetralin, chroman, thiochroman, tetrahydroquinoline, dihydronaphthalene, tetrahydronaphthalen, chromene, thiochromene, dihydroquinoline, indan, dihydrobenzofuran, dihydrobenzothiophene, indene, benzofuran, benzothiophene, coumarin and coumarinone, wherein said aryl is unsubstituted or is substituted with one or two alkyl, alkenyl, alkynyl, aryl, halo, haloalkyl, hydroxyl, alkoxyl, alkylcarbonyl, formyl, oxycarbonyl, carboxyl, alkyl carboxylate, alkyl amide, aminocarbonyl, amino, cyano, diazo, nitro, thio, sulfoxyl, or sulfonyl groups.

7. The method of claim 4 wherein Y is phenyl.

8. The method of claim 4 wherein A is CH₂.

9. The method of claim 8 wherein X is NH.

10. The method of claim 9 wherein n is 0 or an integer of 1 or 2 and R⁴ is selected from the group consisting of methyl, methoxy, fluoro and chloro.

11. The method of claim 10 wherein R¹ is selected from the group consisting of hydrogen, methyl, ethyl and i-propyl.

12. The method of claim 8 wherein R³ is selected from the group consisting of methyl, butyl, phenyl, benzyl, pyridyl, furanylmethylenyl, thienyl and thienyl methylenyl.

13. The method of claim 12 wherein p is 0 or p is 1 and R² is selected from the group consisting of hydroxyl, methoxy, nitro, amino, acetamido and benzyloxy.

14. The method of claim 13 wherein p is 1 and R² is a 5-hydroxy group; R¹ is selected from the group consisting of methyl, ethyl, i-propyl and phenyl; R³ is selected from the group consisting of benzyl, thienylmethylenyl and furanylmethylenyl; n is 1 or 2 and R⁴ is selected from the group consisting of methoxy and fluoro.

15. The method of claim 4 wherein said compound is selected from the group consisting of
1-Benzyl-5-hydroxy-2-methyl-1H-indole-3-carboxylic Acid, 3,5-Difluorobenzylamide;
5-Hydroxy-2-methyl-1-thiophen-2-ylmethyl-1H-indole-3-carboxylic Acid, 3,4-Difluorobenzylamide;
1-Butyl-5-hydroxy-2-methyl-1H-indole-3-carboxylic Acid, 3,5-Difluoro-benzylamide;
1-Furan-2-ylmethyl-5-hydroxy-2-methyl-1H-indole-3-carboxylic Acid, 3,4-Difluorobenzylamide;
5-Hydroxy-2-methyl-1-thiophen-2-ylmethyl-1H-indole-3-carboxylic Acid, 3,5-Difluorobenzylamide;
1-Furan-2-ylmethyl-5-hydroxy-2-methyl-1H-indole-3-carboxylic Acid 3,5-Difluorobenzylamide;
1-Benzyl-5-hydroxy-2-methyl-1H-indole-3-carboxylic Acid. 3,4-Difluoro-benzylamide;
5-Hydroxy-2-methyl-1-thiophen-2-ylmethyl-1H-indole-3-carboxylic Acid, 3-Fluorobenzylamide;
5-Hydroxy-2-methyl-1-thiophen-2-ylmethyl-1H-indole-3-carboxylic Acid, Benzylamide;
5-Hydroxy-2-methyl-1-thiophen-2-ylmethyl-1H-indole-3-carboxylic Acid, 3-Methoxybenzylamide;
1-Butyl-5-hydroxy-2-methyl-1H-indole-3-carboxylic Acid, 3-Methoxy-benzylamide;
5-Hydroxy-2-methyl-1-thiophen-2-ylmethyl-1H-indole-3-carboxylic Acid, 4-Fluorobenzylamide;
5-Hydroxy-2-methyl-1-thiophen-2-ylmethyl-1H-indole-3-carboxylic Acid, 4-Methylbenzylamide;
5-Hydroxy-2-methyl-1-thiophen-2-ylmethyl-1H-indole-3-carboxylic Acid, 3-Chlorobenzylamide;
5-Hydroxy-2-methyl-1-thiophen-2-ylmethyl-1H-indole-3-carboxylic Acid, 4-Chlorobenzylamide;
5-Hydroxy-2-methyl-1-thiophen-2-ylmethyl-1H-indole-3-carboxylic Acid, 2-methoxybenzylamide;
1-Benzyl-2-ethyl-5-hydroxy-1H-indole-3-carboxylic Acid, 3,4-Difluoro-benzylamide;
1-Benzyl-2-ethyl-5-hydroxy-1H-indole-3-carboxylic Acid, 3-Methoxy-benzylamide;
1-Benzyl-5-hydroxy-2-isopropyl-1H-indole-3-carboxylic Acid, 3,4-Difluorobenzamide;
5-Hydroxy-2-methyl-1-phenyl-1H-indole-3-carboxylic Acid 3,4-Difluoro-benzylamide;
5-Hydroxy-2-methyl-1-pyridin-2-yl-1H-indole-3-carboxylic Acid 3,4-Difluoro-benzylamide;
5-Hydroxy-2-methyl-1-thiophen-2-yl-1H-indole-3-carboxylic Acid 3,4-Difluorobenzylamide;
1-Benzyl-2-ethyl-5-hydroxy-1H-indole-3-carboxylic Acid 3,5-Difluoro-benzylamide;
1-Benzyl-5-hydroxy-2-isopropyl-1H-indole-3-carboxylic Acid, 3,5-difluorobenzylamide;
1-Benzyl-5-hydroxy-2-isopropyl-1H-indole-3-carboxylic Acid, 3-methoxybenzylamide; and
1-Benzyl-5-hydroxy-2-phenyl-1H-indole-3-carboxylic Acid, 3,5-Difluoro-benzylamide.

16. The method of claim 15 wherein said compound is selected from the group consisting of
1-Benzyl-5-hydroxy-2-methyl-1H-indole-3-carboxylic Acid, 3,5-Difluorobenzylamide;
1-Furan-2-ylmethyl-5-hydroxy-2-methyl-1H-indole-3-carboxylic Acid 3,5-Difluorobenzylamide;
5-Hydroxy-2-methyl-1-thiophen-2-ylmethyl-1H-indole-3-carboxylic Acid, 3-Methoxybenzylamide;
1-Benzyl-2-ethyl-5-hydroxy-1H-indole-3-carboxylic Acid, 3,4-Difluoro-benzylamide;
1-Benzyl-2-ethyl-5-hydroxy-1H-indole-3-carboxylic Acid 3,5-Difluoro-benzylamide;
1-Benzyl-5-hydroxy-2-isopropyl-1H-indole-3-carboxylic Acid, 3,5-difluorobenzylamide;
1-Benzyl-5-hydroxy-2-isopropyl-1H-indole-3-carboxylic Acid, 3-methoxybenzylamide; and
1-Benzyl-5-hydroxy-2-phenyl-1H-indole-3-carboxylic Acid, 3,5-Difluoro-benzylamide.

17. A method for treating inflammation, the method comprising the step of administering to a patient in need of such treatment a compound represented by the general formula I: 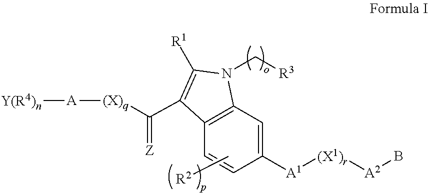
wherein:
R¹, R², R³ and R⁴ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, carbocyclic hydrocarbon groups having from 3 to 20 carbon atoms, heterocyclic groups having up to 20 carbon atoms and at least one of oxygen, nitrogen and/or sulfur in the ring, halo, C₁ to C₁₂ haloalkyl, hydroxyl, C₁ to C₁₂ alkoxy, C₃ to C₂₀ arylalkyloxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, and sulfonyl groups;
X and X¹ are independently selected from the group consisting of NR⁵, O and S;
R⁵ is hydrogen, an alkyl group of 1 to 10 carbons, a cycloalkyl group of 5 to 10 carbons, phenyl or lower alkylphenyl;
Y is a carbocyclic aryl or heterocyclic aryl group wherein said carbocylic aryl comprises from 6 to 20 atoms and said heterocyclic aryl comprises from 2 to 20 carbon atoms and from 1 to 5 heteroatoms selected from the group consisting of nitrogen, oxygen and sulfur, and wherein said aryl may be bonded to A at any position;
Z is O or S;
n is 0 or an integer of from 1 to 5;
o is 0 or an integer of from 1 to 3;
p is 0 or an integer of from 1 to 3;
q is 0 or 1;
r is 0 or 1;
A, A¹ and A² are independently selected from the group consisting of (CH₂)ᵥ wherein v is 0 or an integer of from 1 to 12, branched chain alkyl having 3 to 12 carbons, cycloalkyl having 3 to 12 carbons, alkenyl having 2 to 10 carbons and 1-3 double bonds and alkynyl having 2 to 10 carbons and 1 to 3 triple bonds;
B is selected from the group consisting of hydrogen, OR⁶, COOR⁷, NR⁸R⁹, CONR⁸R⁹, COR¹⁰, CH═NOR¹¹, CH═NNR¹²R¹³, wherein R⁶, R⁷, R¹⁰ and R¹¹ are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, R⁸, R⁹, R¹² and R¹³ are are independently selected from the group consisting of hydrogen, straight or branched chain alkyl having 1 to 12 carbons, alkenyl having 2 to 6 carbons and 1 or 2 double bonds, alkynyl having 2 to 6 carbons and 1 or 2 triple bonds, a carbocyclic hydrocarbon group having from 3 to 20 carbon atoms, a heterocyclic group having up to 20 carbon atoms and at least one of oxygen, nitrogen and/or sulfur in the ring, or R⁸ and R⁹ and/or R¹² and R¹³, together, can form a divalent carbon radical of 2 to 5 carbons to form a heterocyclic ring with nitrogen, wherein any of R⁶, R⁷, R⁸, R⁹, R¹⁰, R¹¹, R¹² or R¹³ may be substituted with one or more halogen, hydroxy, alkyloxy, cyano, nitro, mercapto or thiol radical; provided however, when v is 0, and r is 0, B is not hydrogen; or B is a carbocyclic hydrocarbon group having from 3 to 20 carbon atoms, or a heterocyclic group having up to 20 carbon atoms and at least one of oxygen, nitrogen and/or sulfur in the ring, and wherein when said B is a carbocyclic or heterocyclic group B may be bonded to A² at any position, or a pharmaceutically acceptable salt of said compound.

18. The method of claim 17, wherein the compound is selected from the group consisting of the following compounds:

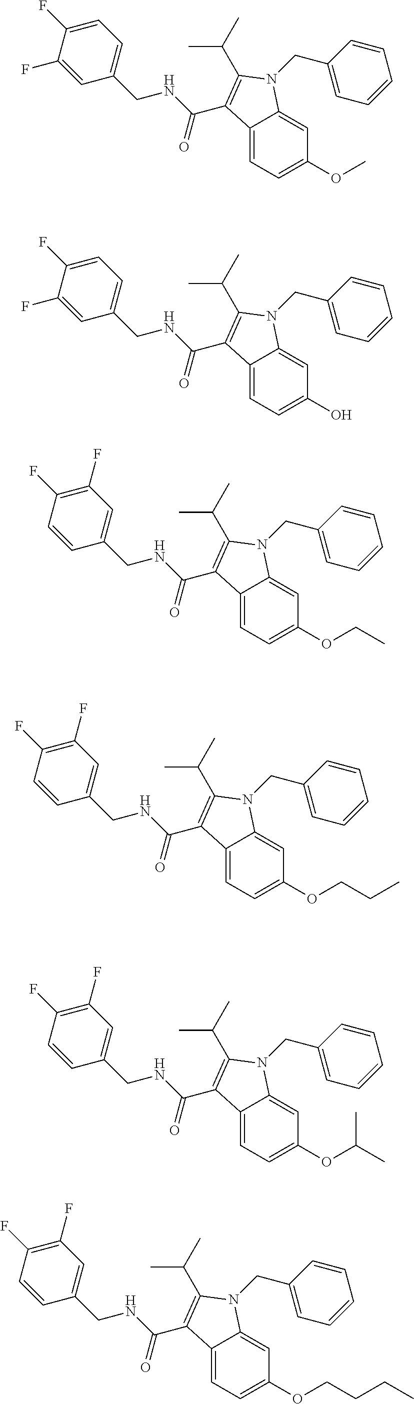

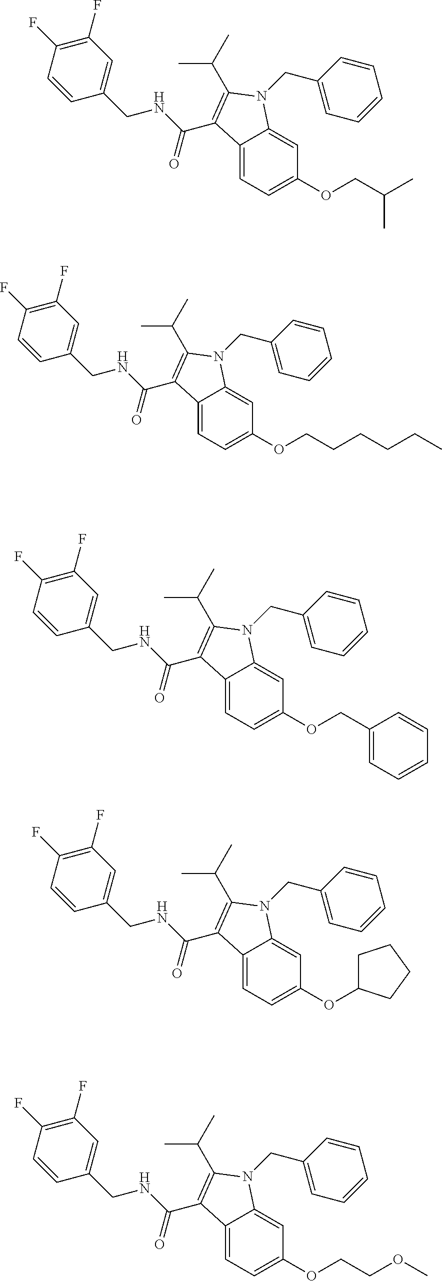

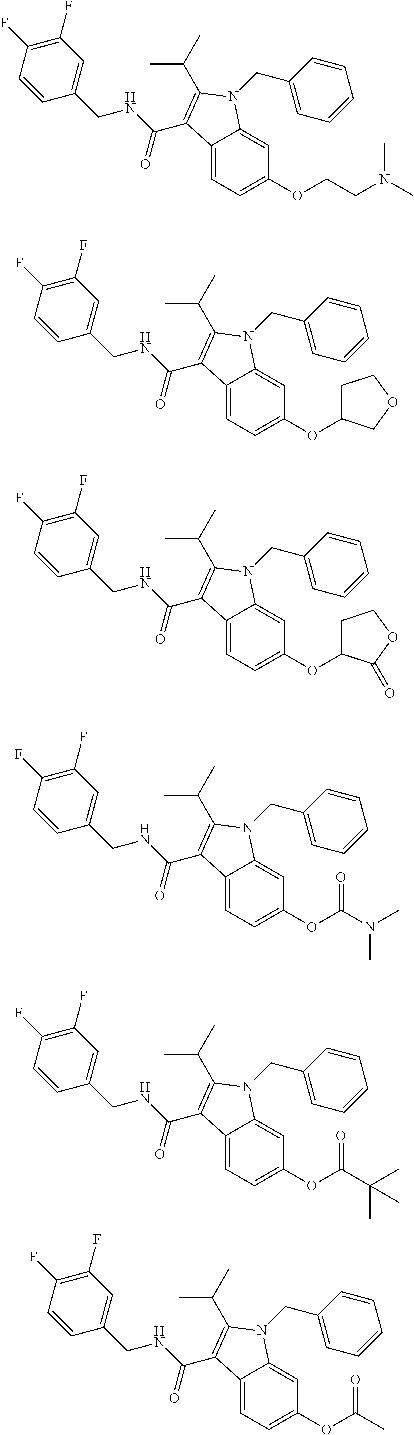

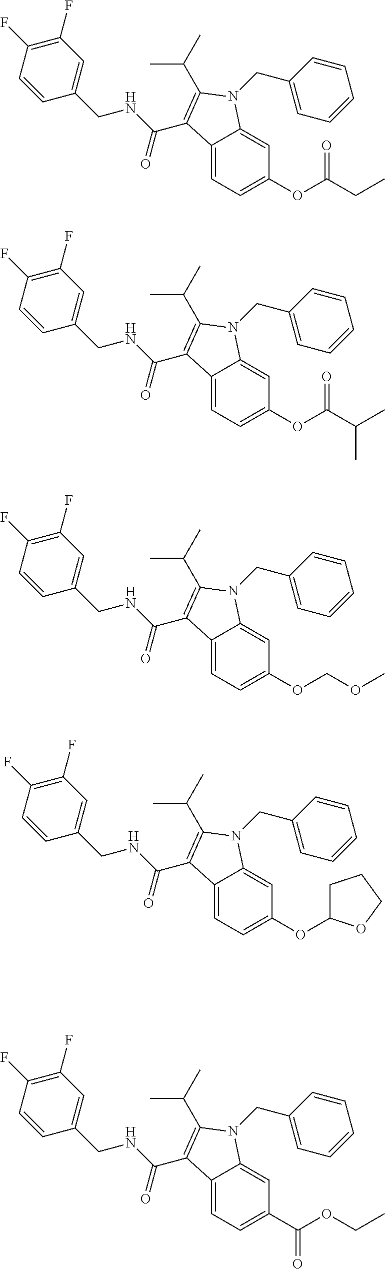

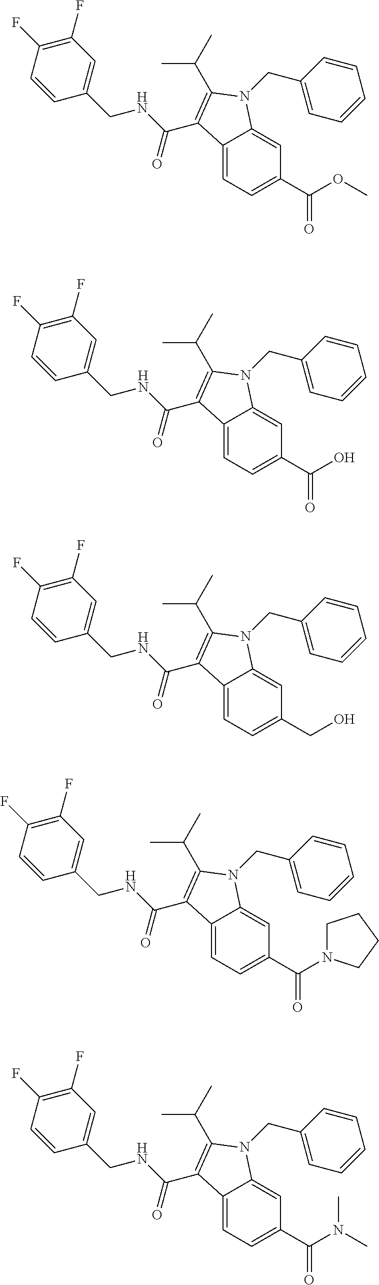

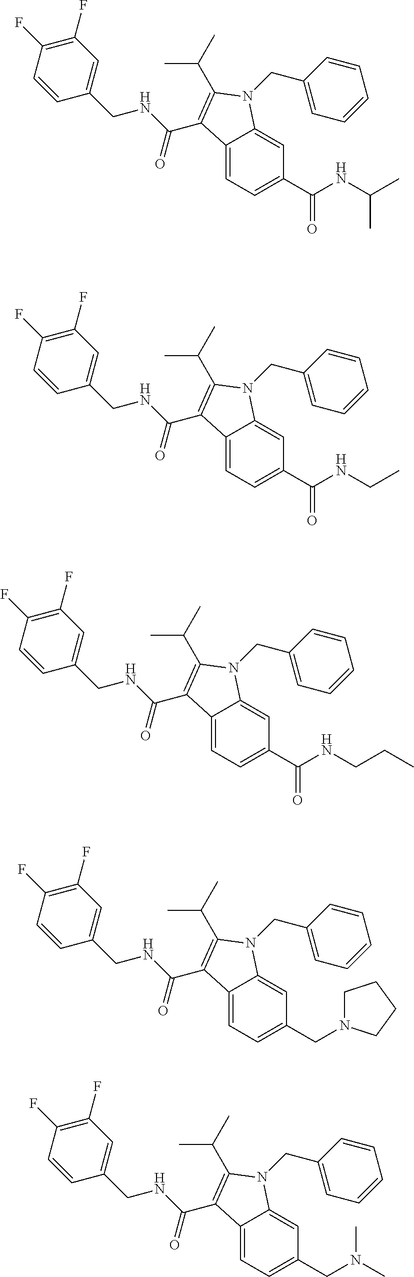

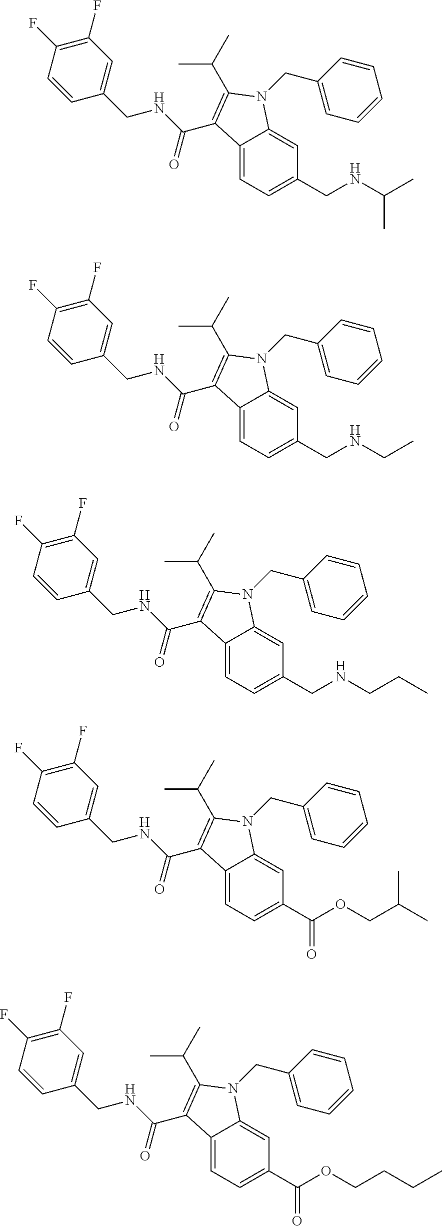

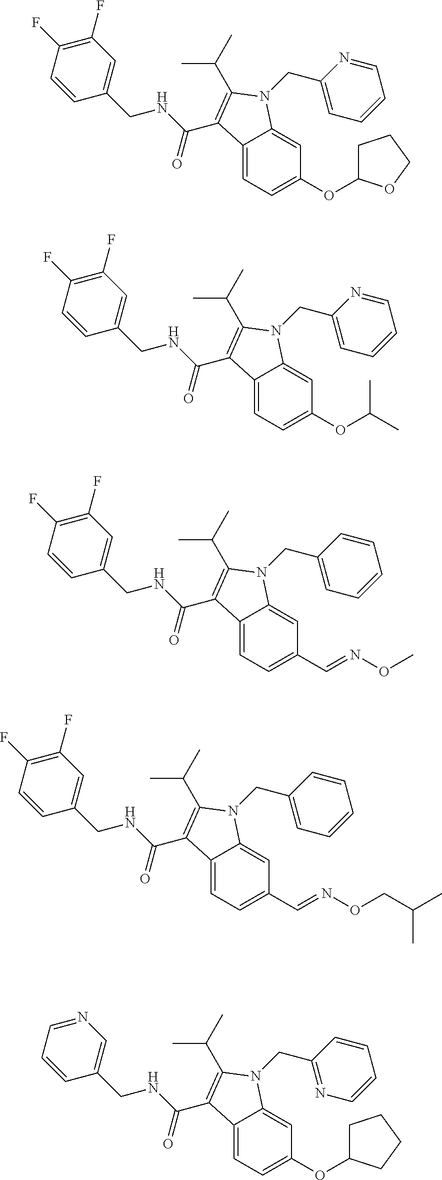

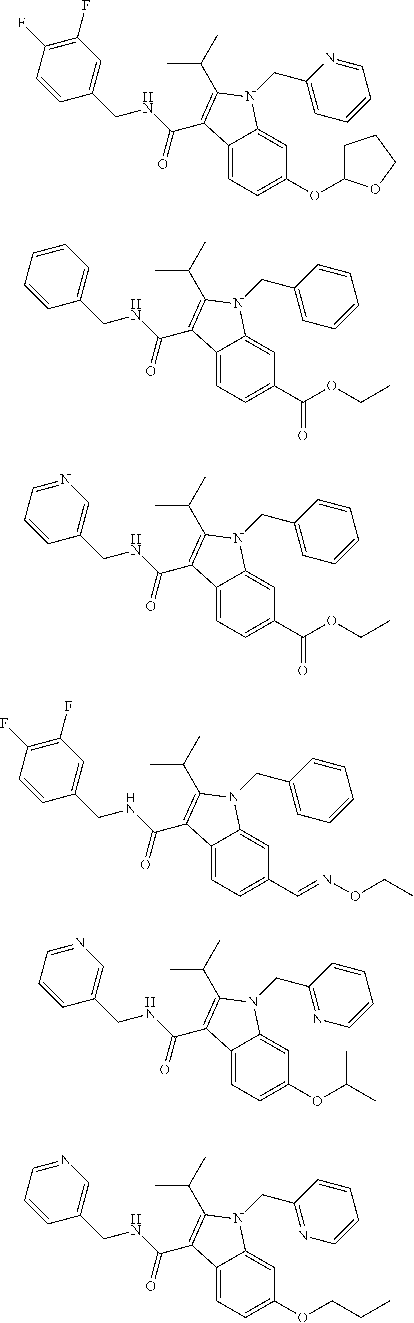

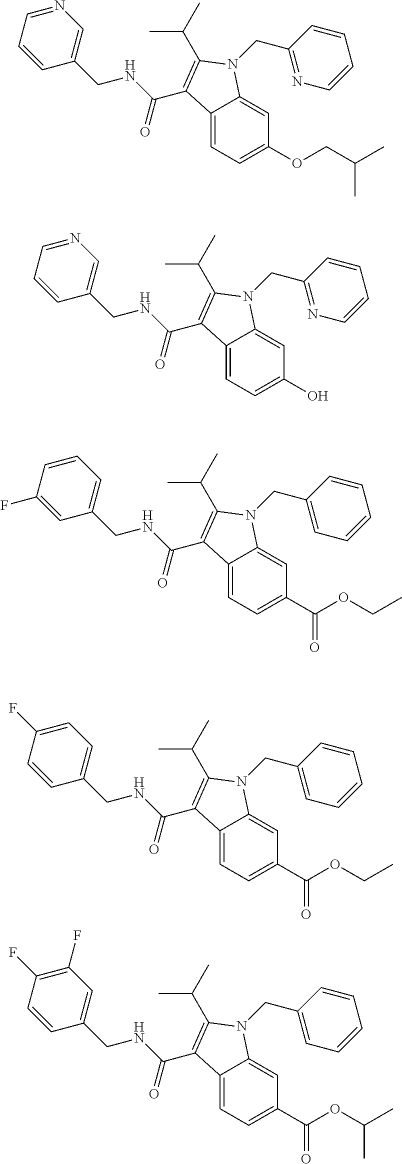

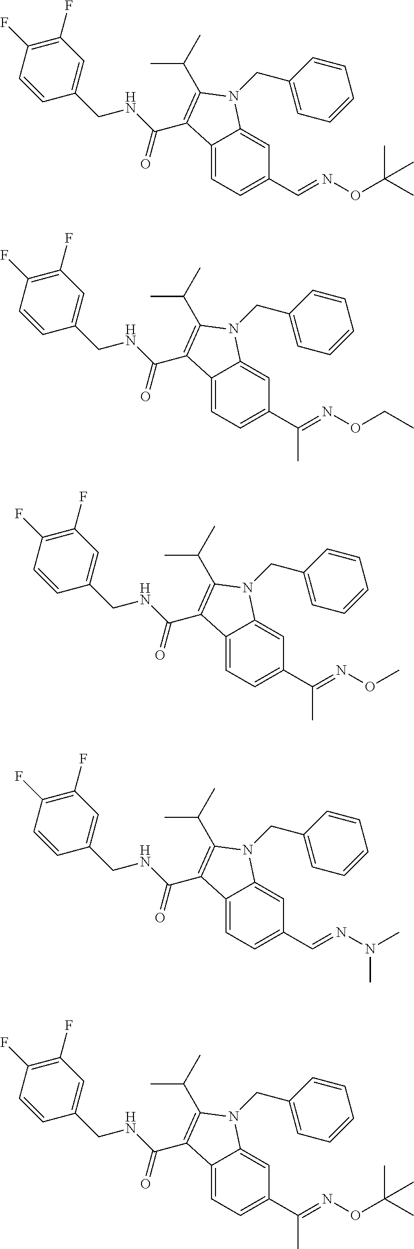

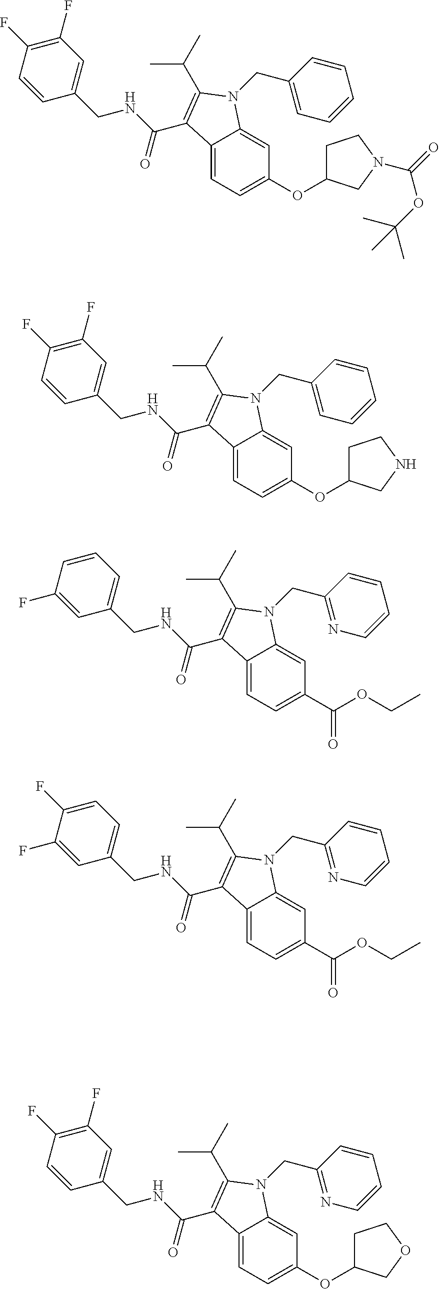

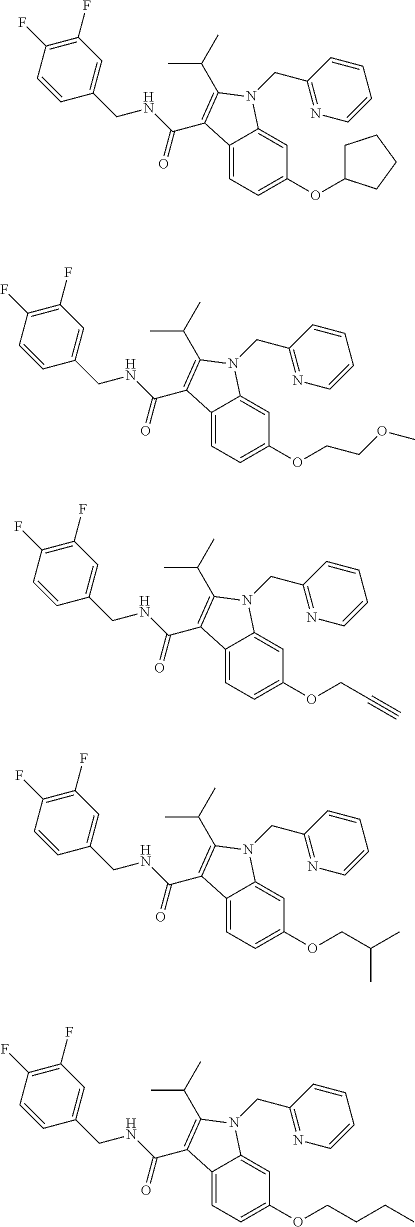

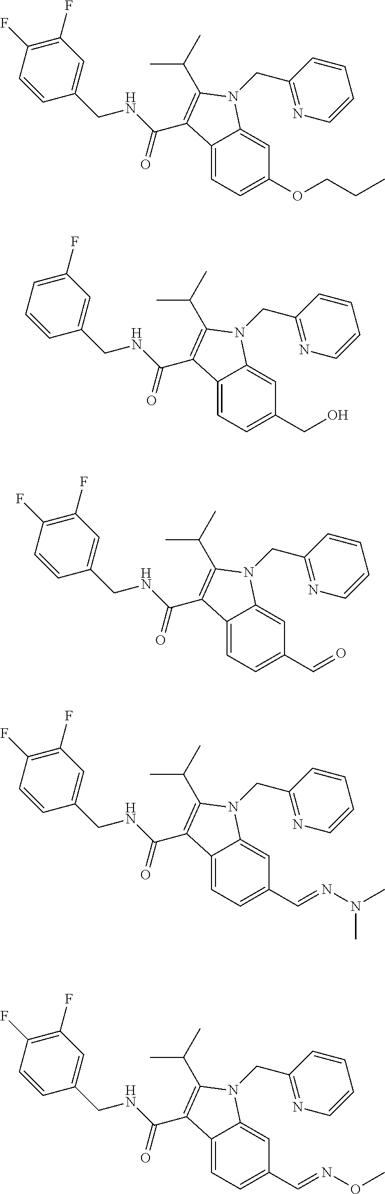

19. A method for treating inflammation, the method comprising the step of administering to a patient in need of such treatment a compound represented by the general formula 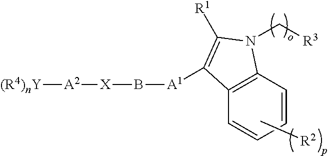
wherein:
A¹ and A² are independently selected from the group consisting of (CH₂)m where m is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;
B is selected from the group consisting of (CH₂)ₙ, where n is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, C═C(R⁵)₂, C═O, C═S, R⁶C═NR⁶, R⁶C═CR⁶, C═NOR⁵, CR⁵OR⁵, C(OR⁶)₂, CR⁶N(R⁶)₂, C(N(R⁵)₂)₂, CR⁵SR⁵, C(SR⁵)₂, SO, SO₂, and heterocyclic aryl comprising from 2 to 14 carbon atoms and from 1 to 3 heteroatoms selected from the group consisting of nitrogen, oxygen and sulfur;
X is selected from the group consisting of (CH₂)r, where r is 0 or an integer of from 1 to 6, lower branched chain alkyl having 2 to 6 carbons, cycloalkyl having 3 to 6 carbons, alkenyl having 2 to 6 carbons and 1 or 2 double bonds, alkynyl having 2 to 6 carbons and having 1 or 2 triple bonds, NR⁵, O and S;
provided that when m is 0 and B is C═O then X is not NR⁵, O or S;
Y is R⁶, or a carbocyclic aryl group comprising from 6 to 14 carbon atoms or a heterocyclic aryl group comprising from 2 to 14 carbon atoms and from 1 to 3 heteroatoms selected from the group consisting of nitrogen, oxygen and sulfur;
o is 0 or an integer of from 1 to 3;
p is 0 or an integer of from 1 to 4;
R¹, R², R³, R⁴ are independently selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxy, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl, sulfonyl, 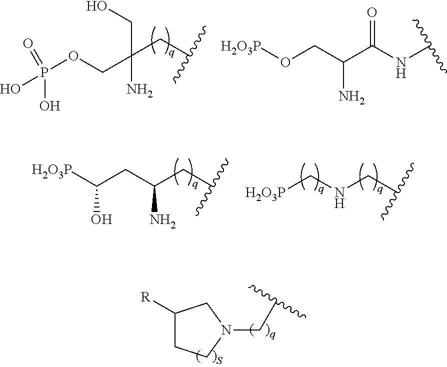
wherein R is CO₂H or PO₃H₂ and q is 0 or an integer of 1 to 5 and s is 0 or an integer from 1 to 3;
R⁵ is selected from the group consisting of hydrogen, straight or branched chain alkyl having 1 to 12 carbons, cycloalkyl having 3 to 6 carbons, alkenyl having 2 to 6 carbons and 1 or 2 double bonds, alkynyl having 2 to 6 carbons and 1 or 2 triple bonds, aryl, halo, C₁ to C₁₂ haloalkyl, hydroxyl, C₁ to C₁₂ alkoxy, C₁ to C₁₂ alkylcarbonyl, formyl, oxycarbonyl, carboxy, C₁ to C₁₂ alkyl carboxylate, C₁ to C₁₂ alkyl amide, aminocarbonyl, amino, cyano, diazo, nitro, thio, sulfoxyl and sulfonyl; and
R⁶ is selected from the group consisting of straight or branched chain alkyl having 1 to 12 carbons, cycloalkyl having 3 to 6 carbons, alkenyl having 2 to 6 carbons and 1 or 2 double bonds and alkynyl having 2 to 6 carbons and 1 or 2 triple bonds.

20. The method of claim 19 wherein said aryl group is selected from the group consisting of benzene, pyridine, pyrazine, pyridazine, pyrimidine, triazine, thiophene, furan, thiazole, thiadiazole, isothiazole, oxazole, oxadiazole, isooxazole, naphthalene, quinoline, tetralin, chroman, thiochroman, tetrahydroquinoline, dihydronaphthalene, tetrahydronaphthalene, chromene, thiochromene, dihydroquinoline, indan, dihydrobenzofuran, dihydrobenzothiophene, indene, benzofuran, benzothiophene, coumarin and coumarinone, which aryl is unsubstituted or is substituted with one or two alkyl, alkenyl, alkynyl, aryl, halo, haloalkyl, hydroxyl, alkoxyl, alkylcarbonyl, formyl, oxycarbonyl, carboxyl, alkyl carboxylate, alkyl amide, aminocarbonyl, amino, cyano, diazo, nitro, thio, sulfoxyl, or sulfonyl groups.

21. The method of claim 20 wherein o is 1 and R³ is phenyl.

22. The method of claim 21 wherein R¹ is i-propyl.

23. The method of claim 22 wherein p is 1 and R² is hydroxy methyloxymethyloxy or dihydropyranyloxy.

24. The method of claim 23 wherein B is selected from the group consisting of C═C(R⁵)₂, C═O and C═NOR⁵.

25. The method of claim 24 wherein Y is R⁶.

26. The method of claim 25 wherein R⁶ is selected from the group consisting of methyl, n-propyl, and i-butyl.

27. The method of claim 22 wherein Y is selected from the group consisting of phenyl and 2,5 difluoro phenyl.

28.-75. (canceled)
